(19) United States
(12) Patent Application Publication
(10) Pub. No.: US 2017/0304649 A1
(43) Pub. Date: Oct. 26, 2017

(54) ELECTRIC CURRENT GENERATING APPARATUS, CONTROL METHOD FOR ELECTRIC CURRENT GENERATING APPARATUS, REAL-TIME TRACKING AND IRRADIATING SYSTEM, X-RAY IRRADIATING APPARATUS, AND CONTROL METHOD FOR X-RAY IRRADIATING APPARATUS

(71) Applicant: KABUSHIKI KAISHA TOSHIBA, Minato-ku (JP)

(72) Inventors: Akio SUMITA, Yokohama (JP); Yasushi Iseki, Yokohama (JP)

(73) Assignee: KABUSHIKI KAISHA TOSHIBA, Minato-ku (JP)

(21) Appl. No.: 15/646,807

(22) Filed: Jul. 11, 2017

Related U.S. Application Data
(63) Continuation of application No. PCT/JP2016/069437, filed on Jun. 30, 2016.

(30) Foreign Application Priority Data
Jul. 1, 2015 (JP) 2015-132874
Aug. 6, 2015 (JP) 2015-156391

(51) Int. Cl. A61N 5/10

(52) U.S. Cl. CPC A61N 5/10; A61N 2005/1097

(57) ABSTRACT

An X-ray irradiating apparatus according to an embodiment is an X-ray irradiating apparatus that can transmit a maintenance electric current for suppressing the motion of a diaphragm in a subject, and includes an electric current outputting unit, electrode units, an electric current output controlling unit and an operating unit. The electric current outputting unit outputs the maintenance electric current for maintaining the contraction of the muscle. The electrode units, which are disposed on a skin surface of the subject, transmit the maintenance electric current. The electric current output controlling unit controls the electric current outputting unit to switch between a state in which the maintenance electric current is output to the electrode units and a state in which the maintenance electric current is not output to the electrode units. The operating unit performs the operation of the electric current output controlling unit.

### CROSS REFERENCE TO RELATED APPLICATIONS

This application is based upon and claims the benefit of priority from the prior Japanese Patent Application No. 2015-132874, filed on Jul. 1, 2015, and the prior Japanese Patent Application No. 2015-156391, filed on Aug. 6, 2015 the entire contents of which are incorporated herein by reference.

### FIELD

Embodiments of the present invention relate to an electric current generating apparatus, a control method for an electric current generating apparatus, a real-time tracking and irradiating system, an X-ray irradiating apparatus, and a control method for an X-ray irradiating apparatus.

### BACKGROUND

A high-accuracy radiation therapy technology of intensively irradiating an affected part target with high-dose rays while protecting normal cells is in widespread clinical use. In the high-accuracy radiation therapy technology, a therapeutic beam such as a heavy particle beam, a proton beam and an X-ray is used, and a stereotactic body radiation therapy, an intensity-modulated radiation therapy and the like are performed. In these radiation therapies, the therapy plan about the energy, radiation dose, incident direction and others of the therapeutic beam is carefully made such that a great killing effect is exerted on tumor cells as the affected part target, and the therapy is performed in accordance with the therapy plan. Meanwhile, organs and the like included in the thoracic cavity and abdominal cavity sandwiching the diaphragm make respiratory movement. By the respiratory movement, these organs sometimes make three-dimensional movements that cannot be tracked through the movement of the body surface. Therefore, in the case in which the affected part target exists in these organs, it is necessary to track the affected part target three-dimensionally, and a real-time tracking and irradiating method is applied to the three-dimensional tracking.

In the real-time tracking and irradiating method, a gating irradiating method in which the position of the affected part target is tracked using a two-orthogonal X-ray fluoroscopic radiographing apparatus is used. That is, in the gating irradiating method, the irradiation with the therapeutic beam is performed when the affected part target is positioned in a gate that is the irradiation range of the therapeutic beam. Further, in the method of tracking the position of the affected part target, a respiratory signal indicating a respiratory waveform is sometimes used. That is, the timing of the irradiation with the therapeutic beam is synchronized with a predetermined phase of the respiratory waveform. These methods relatively reduce the gap between the affected part target making the respiratory movement and the irradiation position of the therapeutic beam, and the therapy can be carried out while the therapy subject performs free respiration. That is, these methods make it possible to decrease an IM (Internal Margin), which is a body margin in consideration of the physiological movement of the affected part target.

However, in the real-time tracking and irradiating method, the therapy effect is sometimes influenced, depending on the repeatability of the respiration. That is, in some cases, the respiratory waveform varies with time, and thereby, the affected part target does not regularly enter the gate for the therapeutic beam, so that the radiation dose is influenced. The respiratory waveform sometimes varies by uncertain factors, for example, when the respiratory phase is changed by the drift phenomenon of the respiration, when the movement locus of the affected part target exhibits a hysteresis loop, or when the respiration stop phase is shifted. Further, in an existing system, there are limitations to the real-time tracking of a constantly moving affected part target. Therefore, there is an element of the prediction of the movement of the affected part target, and accordingly, the repeatability of the respiration, that Is, the repeatability of the respiratory waveform is further important.

Hence, sufficient guidance, instruction and respiration training of respiratory movement measures to the therapy subject are important for obtaining the repeatability of the respiration. As specific methods for improving the repeatability of the respiration, for example, an oxygen suction method of decreasing a respiration rate and a ventilation quantity by sucking oxygen, a respiration stop method of stopping the respiration at the same level spontaneously or passively, an abdominal compression method of fixing the abdomen by a band, a shell or the like, and a regular respiration learning method of regularly performing the respiration using a metronome have been attempted. However, even by using these methods, it is difficult to obtain a desired level of the repeatability of the respiration, and there is a problem of the increase in the burden on the therapy subject.

Further, in the current therapy, it is necessary to set the IM for each therapy subject in consideration of the above-described uncertain factors, and the burden on medical institutions has increased.

Furthermore, also in a general X-ray radiography such as CT and plain radiography, it is necessary to suppress the deterioration in the quality of the radiographed image due to the body movement by the respiration, expiration shortage, inspiration shortage or the like.

Further, in the real-time tracking and irradiating method, the movement of the affected part target is tracked by X-ray fluoroscopy, for example, at a high frequency of 30 fps, and it is demanded to further decrease the Integral dose of the X-ray with which a subject is irradiated.

Hence, embodiments of the present invention have been made in consideration of such points, and have an object to provide an electric current generating apparatus that suppresses the movement of the diaphragm in the subject at a higher accuracy.

Further, embodiments of the present invention have an object to provide an X-ray irradiating apparatus that makes it possible to further decrease the integral dose of the X-ray to be used for tracking the affected part target in the subject.

### BRIEF DESCRIPTION OF THE DRAWINGS

FIG. 1 is a block diagram for explaining a whole configuration of a real-time tracking and irradiating system according to a first embodiment.

FIG. 2 is a block diagram for explaining a configuration of an electric current generating apparatus.

FIG. 3 is a schematic diagram showing a time series change in the height of an abdomen and a time series change in the air quantity in a lung.

FIG. 4 is a schematic diagram showing a respiratory waveform and an output range of an analytic signal.

FIG. 5 is a diagram for explaining a pulsed maintenance electric current that is generated in an electric current generating unit.

FIG. 6 is a schematic diagram showing the position of an abdominal muscle related to respiration and the disposed position of electrode units.

FIG. 7 is a schematic diagram showing a movement range of a tumor and the position of a gate in a 4D CT.

FIG. 8 is a schematic diagram showing a relation between a respiratory waveform and the movement of a tumor that is an affected part target.

FIG. 9 is a diagram showing a control timing of the electric current generating apparatus.

FIG. 10 is a block diagram for explaining a whole configuration of a CT system according to a second embodiment.

FIG. 11 is a schematic diagram showing a time series change in the air quantity in a lung and an output range of an analytic signal according to the second embodiment.

FIG. 12 is a block diagram for explaining a whole configuration of a plain radiographing system 1200 according to a third embodiment.

FIG. 13 is a block diagram for explaining a whole configuration of an X-ray irradiating apparatus 1300 according to a fourth embodiment.

FIG. 14 is a block diagram for explaining a configuration of an electric current generating body unit according to the fourth embodiment.

FIG. 15 is a schematic diagram showing a time series change in the air quantity in a lung and an output timing of an analytic signal according to the fourth embodiment.

FIG. 16 is a schematic diagram showing a movement range of a tumor and the position of a gate in a 4D CT according to the fourth embodiment.

FIG. 17 is a schematic diagram showing a relation between a respiratory waveform and the movement of a tumor that is an affected part target according to the fourth embodiment.

FIG. 18 is a diagram showing a control timing of the X-ray irradiating apparatus.

### DETAILED DESCRIPTION

An X-ray irradiating apparatus according to an embodiment is an X-ray irradiating apparatus that can transmit a maintenance electric current for suppressing the motion of a diaphragm in a subject, and includes an electric current outputting unit, electrode units, an electric current output controlling unit and an operating unit. The electric current outputting unit outputs the maintenance electric current for maintaining the contraction of the muscle related to the motion of the diaphragm by electric stimulus. The electrode units, which are disposed on a skin surface of the subject, transmit the maintenance electric current to the muscle. The electric current output controlling unit controls the electric current outputting unit to switch between a state in which the maintenance electric current is output to the electrode units and a state in which the maintenance electric current is not output to the electrode units. The operating unit performs the operation of the electric current output controlling unit, based on a signal corresponding to the operation.

Embodiments will now be explained with reference to the accompanying drawings.

### First Embodiment

An electric current generating apparatus according to a first embodiment switches between a state in which a maintenance electric current is output for maintaining the contraction of the abdominal muscle related to the motion of the diaphragm by electric stimulus, to the electrode units, and a state in which the maintenance electric current is not output to the electrode units, in accordance with the operation by a subject, and suppresses the motion of the diaphragm in the subject, in accordance with the operation by the subject. The detail will be explained below.

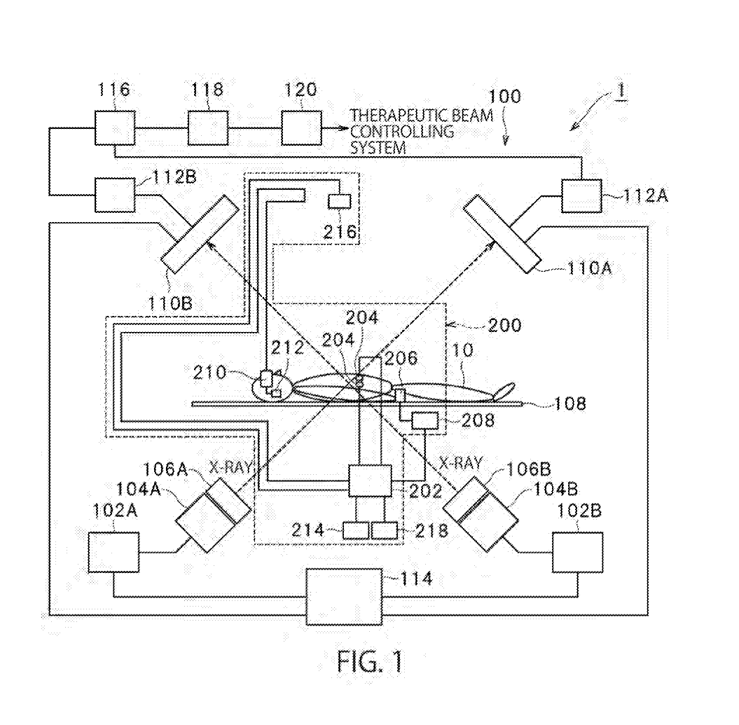

A whole configuration of a real-time tracking and irradiating system 1 according the embodiment will be explained based on FIG. 1 to FIG. 4. FIG. 1 is a block diagram for explaining the whole configuration of the real-time tracking and irradiating system 1 according to the embodiment. As shown in FIG. 1, the real-time tracking and irradiating system 1 according to the embodiment is a system that tracks the position of an affected part target making respiratory movement, and that suppresses the movement of the affected part target by electric stimulus, and is configured to include a real-time tracking and Irradiating apparatus 100 and an electric current generating apparatus 200.

The real-time tracking and irradiating apparatus 100 images the affected part target in a subject 10, using X-rays, and obtains three-dimensional coordinates of the affected part target. That is, the real-time tracking and irradiating apparatus 100 is configured to include a first high-voltage pulse generating unit 102A, a second high-voltage pulse generating unit 102B, a first X-ray tube holding unit 104A, a second X-ray tube holding unit 104B, a first collimator unit 106A, a second collimator unit 106B, a therapy table 108, a first X-ray radiographing unit 110A, a second X-ray radiographing unit 110B, a first 2D image outputting unit 112A, a second 2D image outputting unit 112B, a synchronization controlling unit 114, a 3D image outputting unit 116, a target coordinate outputting unit 118, and an Irradiation permission judging unit 120.

The first high-voltage pulse generating unit 102A generates a first high-voltage pulse. Further, the first X-ray tube holding unit 104A holds an unillustrated first X-ray tube. The first high-voltage pulse is applied to the first X-ray tube, and thereby, the subject 10 is irradiated with a first pulsed X-ray, by the first X-ray tube holding unit 104A. Furthermore, the first collimator unit 106A, which is attached to an X-ray outputting surface of the first X-ray tube, controls the irradiation range of the first pulsed X-ray. On the therapy table 108, the subject 10 lying while facing up is fixed and mounted.

The first X-ray radiographing unit 110A converts the X-ray quantity of the first pulsed X-ray emitted through the first collimator unit 106A into an electric signal, to output it, and is configured by an FPD (Flat Panel Detector) with an indirect conversion scheme, for example. That is, the first X-ray radiographing unit 110A converts the X-ray quantity of the first pulsed X-ray having penetrated the subject 10 into an electric signal, and outputs it. Further, in the first X-ray radiographing unit 110A, a color image intensifier (Color I.I.™) having a higher X-ray sensitivity may be used. In this case, the X-ray irradiation dose necessary for the fluoroscopic radiography is decreased compared to the FPD, and therefore, the X-ray exposure of the subject 10 can be potentially decreased. The first 2D image outputting unit 112A converts the electric signal output by the first X-ray radiographing unit 110A into 2D image data by arithmetic processing, and outputs it.

The second high-voltage pulse generating unit 102B has the same configuration as that of the first high-voltage pulse generating unit 102A, and generates a second high-voltage pulse. Further, the second X-ray tube holding unit 104B has the same configuration as that of the first X-ray tube holding unit 104A, and irradiates the subject 10 with a second X-ray, from a different direction from that of the first X-ray tube holding unit 104A. Furthermore, the second collimator unit 106B also has the same configuration as that of the first collimator unit 106A, and restricts the irradiation range of the second X-ray generated by a second X-ray tube. The second X-ray radiographing unit 110B has the same configuration as that of the first X-ray radiographing unit 110A, and converts the X-ray quantity of a second pulsed X-ray having penetrated the subject 10 into an electric signal, to output it. The second 2D image outputting unit 112B has the same configuration as that of the first 2D image outputting unit 112A, and converts the electric signal output by the second X-ray radiographing unit 110B into two-dimensional image data by arithmetic processing, and outputs it.

Two sets of X-ray fluoroscopic radiographing systems configured by the X-ray emission tube holding units 104A, 104B and the X-ray radiographing units 110A, 110B are disposed so as to be orthogonal across the subject 10. The vertical disposition of the X-ray emission tube holding units 104A, 104B and the X-ray radiographing units 110A, 110B may be reversed, and the two sets of X-ray fluoroscopic radiographing systems may be configured to be inclined by 90° such that the abdominal side and the dorsal side are irradiated with the X-rays.

The synchronization controlling unit 114 performs a control to synchronize the generation timings of the high-voltage pulses in the first high-voltage pulse generating unit 102A and the second high-voltage pulse generating unit 102B. Moreover, the synchronization controlling unit 114 performs a control to synchronize the imaging timings of the first X-ray radiographing unit 110A and the second X-ray radiographing unit 110B with the generation timings of the high-voltage pulses.

The 3D image outputting unit 116 performs a synthesis process of the respective pieces of two-dimensional image data output from the first 2D image outputting unit 112A and the second 2D image outputting unit 112B, to output a three-dimensional image. The target coordinate outputting unit 118 detects the affected part target from the three-dimensional image data based on the respective pieces of two-dimensional image data, and evaluates three-dimensional coordinates. Further, the target coordinate outputting unit 118 may evaluate first two-dimensional coordinates of the affected part target based on the two-dimensional image data output from the first 2D image outputting unit 112A, may evaluate second two-dimensional coordinates of the affected part target based on the two-dimensional image data output from the second 2D image outputting unit 112B, and may evaluate the three-dimensional coordinates of the affected part target based on the first two-dimensional coordinates and the second two-dimensional coordinates.

The irradiation permission judging unit 120 judges the Irradiation permission for a therapeutic beam, based on the three-dimensional coordinates of the affected part target. That is, the irradiation permission judging unit 120 judges whether the affected part target is positioned in a gate that is the irradiation range of the therapeutic beam. Here, in the embodiment, the first X-ray tube holding unit 104A configures a first X-ray irradiating unit, the second X-ray tube holding unit 104B configures a second X-ray irradiating unit, the first X-ray radiographing unit 110A and the first 2D image outputting unit 112A configure a first X-ray imaging unit, the second X-ray radiographing unit 110B and the second 2D image outputting unit 112B configure a second X-ray imaging unit, and the target coordinate outputting unit 118 configures a position detecting unit.

The organ movement includes respiratory movement and heartbeat (in several seconds), includes swallowing and enteric peristalsis (in minutes), and includes the change in the collection quantity of urine in the urinary bladder and the contents in the stomach and intestines (daily change), for example. Therefore, just for the error effect during the irradiation with the therapeutic beam, it is thought that the respiratory movement and the heartbeat are targeted and the repeatability of the heartbeat at rest is high. For this reason, during the irradiation with the therapeutic beam, the measure against the respiratory movement is demanded.

Next, the electric current generating apparatus 200 suppresses the motion of the diaphragm in the subject 10, by transmitting the maintenance electric current for maintaining the contraction of the abdominal muscle related to the motion of the diaphragm, to the abdominal muscle. That is, the electric current generating apparatus 200 is configured to include an electric current generating unit 202, electrode units 204, a push button unit 206, a manual switch unit 208, a respiratory waveform displaying unit 210, a speaker unit 212, an image displaying unit 214, a respiration monitor unit 216 and an inputting unit 218.

The electric current generating unit 202 generates the maintenance electric current for maintaining the contraction of the abdominal muscle related to the motion of the diaphragm, and outputs an electric signal corresponding to an input signal. The electrode units 204, which are disposed on the skin surface of the subject 10, transmit the maintenance electric current generated by the electric current generating unit 202, to the abdominal muscle related to the motion of the diaphragm. That is, the electrode units 204 are disposed and fixed at skin surface positions allowing for the stimulus of the abdominal muscle including a rectus abdominis, an obliquus externus abdominis, an obliquus internus abdominis and a transverse abdominal muscle. Further, the electrode units 204 are disposed and fixed at positions of a human-body skin surface outside the X-ray fluoroscopic region. That is, the electrode units 204 are disposed and fixed at positions outside the irradiation ranges of the X-rays that are emitted by the first X-ray tube holding unit 104A and the second X-ray tube holding unit 104B. For example, the electrode unit 204 is configured by an attachment pad that is attached to the human-body skin surface. Further, the electrode unit 204 is configured, for example, by a conductive tape or a conductive film.

The push button unit 206 outputs an ON signal when being depressed. For example, the push button unit 206 is configured as a structure in which the push button unit 206 is fit in a hand of the subject 10 and a button is at the position of the thumb or the like. The manual switch unit 208, which is connected with the push button unit 206, outputs a depression signal for turning on the maintenance electric current, to the electric current generating unit 202, in accordance with the depression operation of the push button unit 206 by the subject 10. That is, the manual switch unit 208 continuously outputs the depression signal during a period in which the push button unit 206 is being depressed by the subject 10. Based on the depression signal, the electric current generating unit 202 generates the maintenance electric current, and outputs it to the electrode units 204. In the embodiment, the push button unit 206 and the manual switch unit 208 configure the operating unit.

The respiratory waveform displaying unit 210 displays a respiratory waveform, in accordance with the input signal from the electric current generating unit 202. Further, when the subject 10 is in a previously set respiratory state, the respiratory waveform displaying unit 210 displays a marker for giving an instruction to depress the push button unit 206, together with the respiratory waveform. Here, the position of the gate for the therapeutic beam is set based on the position of the affected part target in the previously set respiratory state.

When the subject 10 is the previously set respiratory state, the speaker unit 212 generates a sound that is audible for the subject 10, in accordance with the input signal from the electric current generating unit 202. The speaker unit 212 is disposed such that the subject 10 easily listens, and for example, the speaker unit 212 is configured by an earphone that can be fixed to an ear. Further, the tone color to be generated by the speaker unit 212 is not forcible, and is a soft tone color. In the embodiment, the speaker unit 212 configures a sound generating unit.

The image displaying unit 214 displays a picture signal input from the electric current generating unit 202, in response to the depression of the push button unit 206. Thereby, an operator can confirm the depression of the push button unit 206.

The respiration monitor unit 216 acquires a measurement signal related to the respiration waveform, from the subject 10, and outputs it to the electric current generating unit 202. For example, the measurement signal indicates the height of the abdomen. In the respiration monitor unit 216, a non-contact sensor and a contact sensor can be used. For example, as the non-contact sensor, an infrared sensor, an ultrasonic sensor, an electric wave sensor, a laser sensor and the like can be used in the respiration monitor unit 216. On the other hand, as the contact sensor, a piezoelectric sensor, a strain gauge sensor, a servo sensor and the like can be used in the respiration monitor unit 216. Generally, depending on the therapeutic area, the contact sensor sometimes interferes with the irradiation or the fluoroscopy, and is easily influenced by radioactive rays. Therefore, in the example of the embodiment, the non-contact sensor is used.

Further, as the measurement signal, the respiration monitor unit 216 may acquire the ventilation flow quantity in the lung field of the subject 10. The respiratory waveform herein means the time series change in the air quantity in the lung field. That is, the respiratory waveform is the time series change in the integrated value of the ventilation flow quantity. In the embodiment, the respiration monitor unit 216 configures a measuring unit.

The inputting unit 218 inputs an intensity signal indicating the intensity of the maintenance electric current, in accordance with the operation by the operator. The intensity signal is output to the electric current generating unit 202, and the intensity of the maintenance electric current is controlled.

Further, the inputting unit 218 inputs a selection signal for selecting an operation mode of transmitting the maintenance electric current to the abdominal muscle of the subject 10, in accordance with the operation by the operator. The selection signal is output to the electric current generating unit 202, so that the generation of the maintenance electric current is controlled. That is, the inputting unit 218 is used for selecting one of a first mode of outputting the maintenance electric current from the electrode units 204 when the subject 10 depresses the button, a second mode of restricting or stopping the output of the maintenance electric current from the electrode units 204 when the subject 10 is not in the previously set respiratory state, and a third mode of outputting the maintenance electric current from the electrode units 204 when the subject 10 is in the previously set respiratory state. The third mode is a mode of automatically outputting the maintenance electric current from the electrode units 204 regardless of whether the subject 10 performs the depression. For example, the third mode is used in the case of radiographing a patient with a decreased level of consciousness or a patient such as an infant.

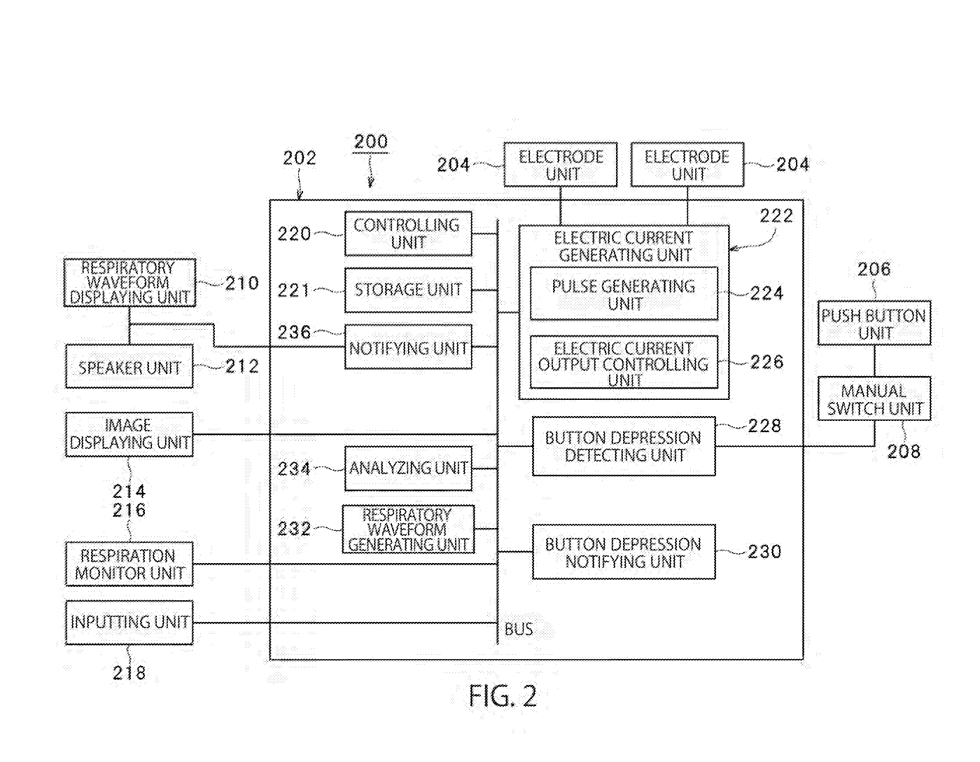

Next, a configuration of the electric current generating unit 202 will be explained based on FIG. 2, with reference to FIG. 1. FIG. 2 is a block diagram for explaining the configuration of the electric current generating apparatus 200. As shown in FIG. 2, the electric current generating unit 202 provided in the electric current generating apparatus 200 is configured to include a controlling unit 220, a storage unit 221, an electric current generating unit 222, a button depression detecting unit 228, a button depression notifying unit 230, a respiratory waveform generating unit 232, an analyzing unit 234, and a notifying unit 236.

The controlling unit 220 controls each constituent unit of the electric current generating unit 202 through a bus. That is, the controlling unit 220 is configured by a CPU, for example, and can control each constituent unit by the execution of a program. The storage unit 221 stores therein a control program that is executed by the controlling unit 220, and provides a work area in the program execution by the controlling unit 220.

The electric current generating unit 222 generates the maintenance electric current for maintaining the contraction of the abdominal muscle related to the motion of the diaphragm by electric stimulus. That is, the electric current generating unit 222 is configured to include a pulse generating unit 224 and an electric current output controlling unit 226.

The pulse generating unit 224 generates a pulsed electric current as the maintenance electric current. That is, the pulse generating unit 224 generates a pulsed electric current in which the pulse generation interval is an Interval at which the contraction of the abdominal muscle is maintained.

The electric current output controlling unit 226 controls the pulse generating unit 224. That is, the electric current output controlling unit 226 controls the generation of the pulsed electric current and the Intensity of the pulsed electric current in the pulse generating unit 224, in accordance with the action mode selected by the selection signal from the inputting unit 218.

When the depression signal is detected, the button depression detecting unit 228 outputs an output signal. That is, the button depression detecting unit 228 continuously outputs the output signal to the electric current output controlling unit 226, during a period in which the depression signal is being detected. In response to the input of the output signal, the electric current output controlling unit 226 performs such a control that the pulse generating unit 224 generates the maintenance electric current. In the embodiment, the pulse generating unit 224 configures the electric current outputting unit.

The button depression notifying unit 230 generates a picture signal Indicating the depression state of the button, in accordance with the input of the output signal of the button depression detecting unit 228. Then, the button depression notifying unit 230 outputs the picture signal to the image displaying unit 214, and thereby, displays a picture indicating the depression state of the button, on the image displaying unit 214.

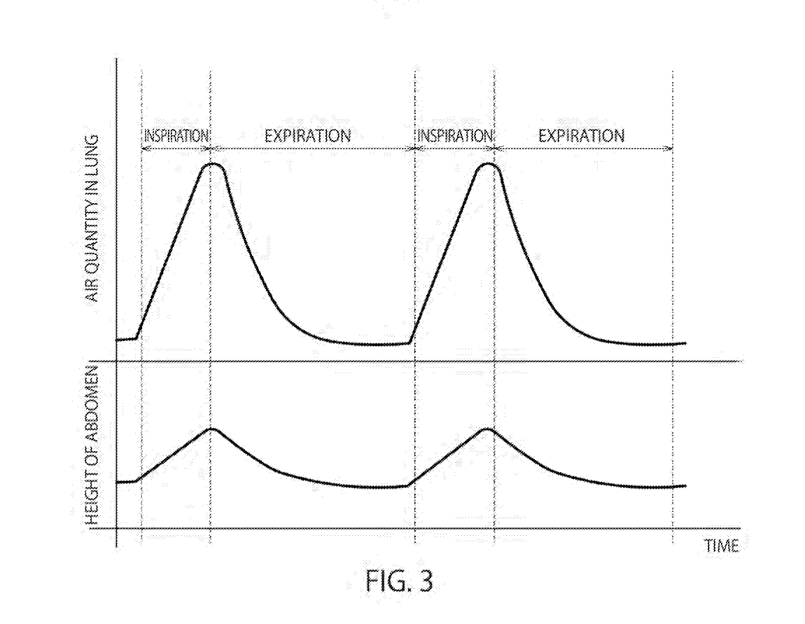

The respiratory waveform generating unit 232 generates the respiratory waveform, based on the information about the height of the abdomen that is acquired by the respiration monitor unit 216. The detail of the generation process in the respiratory waveform generating unit 232 will be explained based on FIG. 3.

FIG. 3 is a schematic diagram showing a time series change in the height of the abdomen and a respiratory waveform. The abscissa in FIG. 3 indicates time, the ordinate in the upper diagram indicates the air quantity in the lung, and the ordinate in the lower diagram Indicates the height of the abdomen measured by the respiration monitor unit 216. As shown in FIG. 3, there is a high correlation between the height of the abdomen measured by the respiration monitor unit 216 and the time series change in the air quantity in the lung.

Generally, the time series change in the air quantity in the lung, that is, the respiratory waveform roughly monotonically increases from the start of the inspiration, and roughly monotonically decreases from the start of the expiration to the end. Similarly, the height of the abdomen roughly monotonically increases in the corresponding inspiration period, and roughly monotonically decreases in the expiration period. Here, the data in FIG. 3 is the example data when the subject 10 is lying at rest while facing up.

Thereby, the respiratory waveform generating unit 232 generates the respiratory waveform, using a previously obtained relation between the time series change in the height of the abdomen and the time series change in the air quantity in the lung field. For example, the respiratory waveform generating unit 232 previously generates a function in which the input value is the height of the abdomen in the inspiration period and the output value is the air quantity in the lung field. Similarly, the respiratory waveform generating unit 232 previously generates a function in which the input value is the height of the abdomen in the expiration period and the output value is the air quantity in the lung field. Thereby, using these functions, the respiratory waveform generating unit 232 converts the height of abdomen into the air quantity in the lung field, and generates the respiratory waveform. That is, the respiratory waveform generating unit 232 generates the respiratory waveform, based on the measurement signal indicating the height of the abdomen measured by the respiration monitor unit 216. Here, in the case in which the ventilation flow quantity is used as the measurement signal, the respiratory waveform generating unit 232 calculates the Integrated value of the ventilation flow quantity, and generates the time series change in the calculated value, as the respiratory waveform.

Further, in the case in which there is a high correlation between the time series change in the height of the abdomen and the time series change in the air quantity in the lung, the respiratory waveform may be generated by the linear conversion of the value of the height of the abdomen. Alternatively, the time series change in the height of the abdomen may be used as the respiratory waveform.

The relation between the time series change in the height of the abdomen and the time series change in the air quantity in the lung is obtained based on the Information of a 4D CT image (Four-Dimensional Computed Tomography). The 4D CT image is imaged in a state in which the subject 10 performs free respiration. Further, when the 4D CT image is imaged, the subject 10 is fixed on the therapy table 108 in a rest state in which the subject 10 is lying while facing up. In this case, the height of the abdomen, which is a distance from the therapy table 108 to a specified region on the abdominal surface, can be evaluated based on the 4D CT image. That is, a CT cross sectional view of the abdomen of the subject 10 that crosses the specified region is taken out of the 4D CT in a time series manner, and the distance from the therapy table 108 to the specified region is calculated as the height of the abdomen. Thereby, it is possible to evaluate the time series change in the height of the abdomen that changes with the lapse of the imaging time when the 4D CT image is imaged.

Meanwhile, by counting the number of voxels in a 4D CT image having a CT value corresponding to the lung field, it is possible to evaluate the volume of the lung field, that is, the air quantity in the lung at the imaging time. That is, based on the 4D CT image, the number of voxels in a 4D CT image having a CT value corresponding to the lung field is counted in a time series manner. Thereby, it is possible to obtain the time series change in the air quantity in the lung that changes with the lapse of the imaging time when the 4D CT image is imaged, that is, the respiratory waveform. In this way, it is possible to previously evaluate the relation between the time series change in the air quantity in the lung, that is, the respiratory waveform, and the time series change in the height of the abdomen.

Again, as shown in FIG. 2, the analyzing unit 234 outputs an analytic signal, based on the respiratory waveform generated by the respiratory waveform generating unit 232. More specifically, the analyzing unit 234 continuously outputs the analytic signal while the air quantity in the lung field is equal to or less than a previously set threshold value. In the case of the radiography in the inspiration state, the analyzing unit 234 continuously outputs the analytic signal while the air quantity in the lung field is equal to or more than a previously set threshold value.

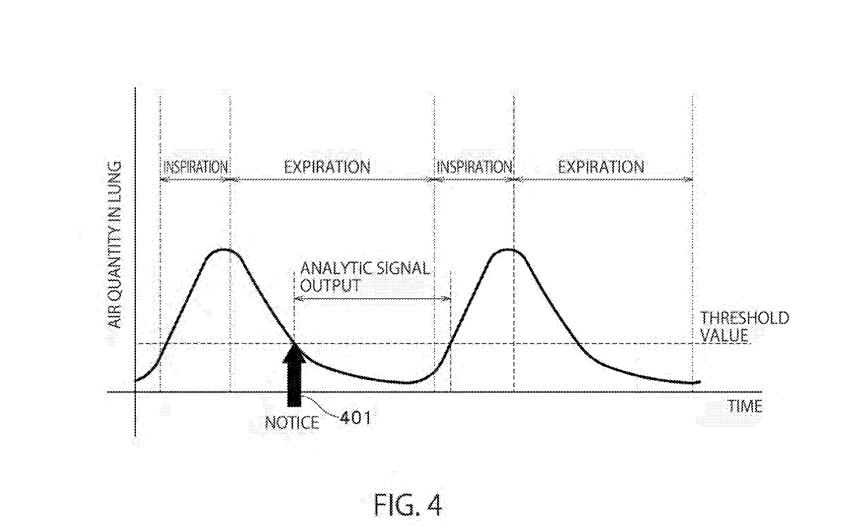

The detail of the analyzing process in the analyzing unit 234 will be explained based on FIG. 4.

FIG. 4 is a schematic diagram showing a time series change in the air quantity in the lung and an output range of the analytic signal. The abscissa in FIG. 4 indicates time, and the ordinate in the figure indicates the air quantity in the lung. Here, a case of outputting the analytic signal when the air quantity in the lung field is equal to or less than the previously set threshold value will be explained.

As shown in FIG. 4, the analyzing unit 234 outputs the analytic signal, when the value of the respiratory waveform generated by the respiratory waveform generating unit 232 is equal to or less than the threshold value, that is, when the air quantity in the lung is equal to or less than the threshold value. The threshold value is set, for example, based on the value of the air quantity in the lung at the time of the maximal expiration, that is, the value of the respiratory waveform at the time of the maximal expiration, and set, for example, to a value indicating a predetermined ratio of the air quantity in the lung at the time of the maximal expiration. The threshold value is set, for example, based on the information of a preliminarily radiographed 4D CT, and the predetermined ratio is 20%, for example.

The respiratory state of the subject 10 in the period of the threshold value or less is a state in which the diaphragm has been relaxed, and is a state in which almost all the air in the lung to be expired at rest has been expired. That is, based on the value of the respiratory waveform at the maximal expiration, the analyzing unit 234 outputs the analytic signal indicating a previously set respiratory state. Here, there is a high correlation between the change quantity of the value of the respiratory waveform with respect to time and the movement quantity of the affected part target making the respiratory movement with respect to time. Therefore, during the period in which the analytic signal is being output, the movement quantity of the affected part target making the respiratory movement with respect to lapse time is also further decreased. As understood from this, the analyzing unit 234 may output the analytic signal, when the change quantity of the value of the respiratory waveform with respect to time becomes equal to or less than a predetermined value. That is, the analyzing unit 234 may set the threshold value, based on the change quantity of the value of the respiratory waveform with respect to time.

Further, a marker 401 is an exemplary marker for the instruction of the depression of the push button unit 206 described above, and is displayed on the respiratory waveform displaying unit 210, based on the timing when the analyzing unit 234 starts the output of the analytic signal.

Again, as shown in FIG. 2, the notifying unit 236 gives a notice that the subject 10 is in the previously set respiratory state. That is, based on the analytic signal output by the analyzing unit 234, the notifying unit 236 displays the marker 401 exemplified in FIG. 4 and the corresponding respiratory waveform, on the respiratory waveform displaying unit 210. Further, the speaker unit 212 outputs a sound signal at the timing of the display of the marker 401. Thereby, the subject 10 is notified that the respiratory state of the subject 10 is the predetermined set respiratory state. The notifying unit 236 may display the marker 401 during a predetermined period from the timing when the analyzing unit 234 outputs the analytic signal, and may continuously display the marker 401 during the period in which the analytic signal is being output.

The whole configuration of the real-time tracking and irradiating system 1 according to the embodiment has been explained above. Next, a control behavior of the electric current output controlling unit 226 will be explained.

When the first mode is selected, the maintenance electric current can be output from the electrode units 204, by the depression of the push button unit 206 by the subject 10. That is, while the output signal is being input from the button depression detecting unit 228, the electric current output controlling unit 226 performs such a control that the pulse generating unit 224 generates the maintenance electric current. Here, the subject 10 can depress the push button unit 206 at the timing of the previously set respiratory state, in accordance with the notice from the notifying unit 236.

When the second mode is selected, the same control behavior as the first mode is performed, and therewith, in the case in which the subject 10 is not in the previously set respiratory state, the output of the maintenance electric current from the electrode units 204 is restricted or prohibited. That is, when the second mode is selected, the electric current output controlling unit 226 controls the pulse generating unit 224 such that the maintenance electric current is generated, in the case in which the above-described output signal is being input and where the analytic signal indicating the previously set respiratory state is being input from the analyzing unit 234. Thereby, it is possible to avoid the motion of the diaphragm from being suppressed in the case in which the subject 10 is not in the previously set respiratory state. Further, when the second mode is selected, by the depression of the push button unit 206, the maintenance electric current is automatically output from the electrode units 204 in the case of being in the previously set respiratory state. Therefore, the subject 10 need not match the push timing of the push button unit 206 with the notice from the notifying unit 236.

When the third mode is selected, the maintenance electric current is output from the electrode units 204 in the case in which the subject 10 is in the previously set respiratory state. That is, when the third mode is selected, the electric current output controlling unit 226 controls the pulse generating unit 224 such that the maintenance electric current is generated, in the case in which the analytic signal indicating the previously set respiratory state is being input from the analyzing unit 234. Thereby, it is possible to suppress the motion of the diaphragm in the case in which the subject 10 is in the previously set respiratory state.

Further, when the second mode or the third mode is selected, the electric current output controlling unit 226 performs such a control that the pulse generating unit 224 outputs the maintenance electric current in a previously set period. Thereby, safety is secured. More specifically, the analyzing unit 234 analyzes the time of the previously set respiratory state, based on the respiratory waveform in the state in which the maintenance electric current is not output from the electrode units 204. The electric current output controlling unit 226 performs such a control that the pulse generating unit 224 outputs the maintenance electric current in a period that is predetermined times longer than that time.

Here, an unillustrated switching element may be disposed between the electrode units 204 and the pulse generating unit 224. In this case, the electric current output controlling unit 226 may stop the maintenance electric current by cutting off the switching element. Thereby, it is possible to cut off the maintenance electric current, even in a state in which the pulse generating unit 224 is driven. Further, it is possible to deal even at the time of an abnormal behavior of the pulse generating unit 224.

As understood from this, whichever of the first mode and the second mode is selected through the inputting unit 218, the subject 10 can give priority to his or her physical condition. That is, for example, when the rhythm of the respiration is unstable, the subject 10 need not push the push button unit 206, and can avoid the maintenance electric current from being output into the body. Therefore, the subject 10 can reflect his or her intention or condition in the output of the maintenance electric current from the electrode units 204.

Further, when the third mode is selected through the inputting unit 218, the third mode is useful even in the case in which a patient with a decreased level of consciousness, an infant or the like has difficulty performing the operation by himself or herself.

Further, the maintenance electric current may be continuously output from the electrode units 204 while the subject 10 is depressing the push button unit 206. Alternatively, the time during which the maintenance electric current is continuously output from the electrode units 204 may be previously set to a predetermined time. In the case of setting the predetermined time, the electric current output controlling unit 226 stops the generation of the maintenance electric current after the lapse of the predetermined time, even when the depression of the push button unit 206 is continued. The predetermined time can be set based on the physical condition of the subject 10, the above-described respiratory waveform before the start of the therapy, and the like. Here, it is preferable that the predetermined time be set in the therapy plan, because of the association with the time of the irradiation with the therapeutic beam.

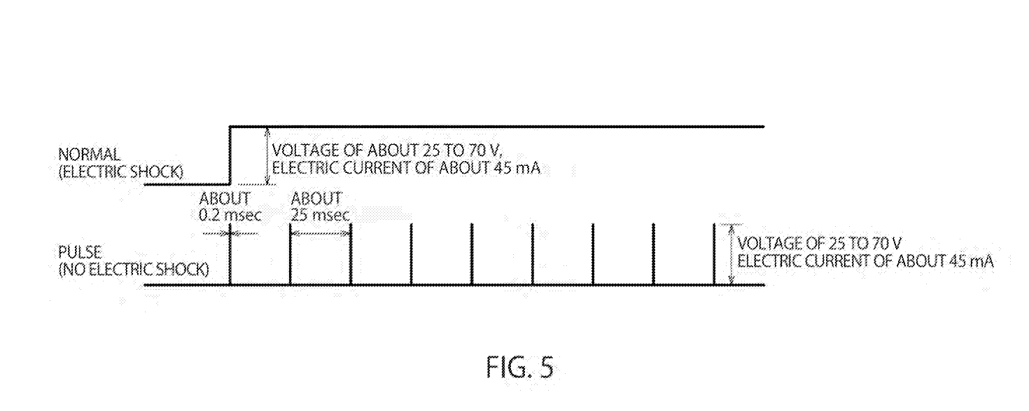

Next, the maintenance electric current that is generated by the electric current generating unit 222 will be explained based on FIG. 5. FIG. 5 is a diagram for explaining the pulsed maintenance electric current that is generated by the electric current generating unit 222. That is, as shown in FIG. 5, the maintenance electric current is a pulsed electric current with a pulse form. In the maintenance electric current, for example, the pulse interval is about 25 msec, and the pulse width is about 0.2 msec. Further, the voltage between the electrode units 204 is 25 to 70 V, for example, and the maintenance electric current to flow between the electrode units 204 is about 45 mA.

Here, the reaction of a muscle tissue of the subject 10 to electric stimulus will be explained. While the maintenance electric current is being transmitted from the skin surfaces or the like to the muscle tissue, the contraction of the muscle tissue is maintained. On the other hand, when the transmission of the maintenance electric current to the muscle tissue is stopped, the muscle tissue is relaxed.

Further, generally, when the maintenance electric current is continuously transmitted to the muscle tissue and the contraction of the muscle tissue is maintained, it is impossible to relax the muscle tissue in the contraction state, by human Intention. Further, as shown in the upper stage of FIG. 5, when the maintenance electric current is transmitted to the muscle tissue as a continuous electric current, the subject 10 gets electric shock, and the subject 10 feels a pain sense. However, when the maintenance electric current is transmitted as a pulsed electric current shown in the lower stage of FIG. 5, the tolerance of the human body to electricity is improved, and the contraction of the muscle tissue is maintained without a pain sense. Therefore, the generation Interval of the pulsed electric current that is generated by the electric current generating unit 222 is an interval allowing the subject not to feel a pain sense and allowing the contraction of the muscle tissue to be maintained. As understood from this, by controlling the maintenance electric current that is output from the electrode units 204, it is possible to temporally control the continued contraction and the relaxation for an aimed muscle tissue, without giving a pain sense to the subject 10.

For reference, such a mechanism for temporally controlling the contraction and relaxation of the muscle tissue is generally used in low-frequency therapy devices and electric therapy devices, for example. As for handling, these therapy devices can be handled by general persons having no medical license. Further, these therapy devices are configured at low cost, because the maintenance electric current can be oscillated by an electric power comparable to a dry battery and furthermore the structure is simple.

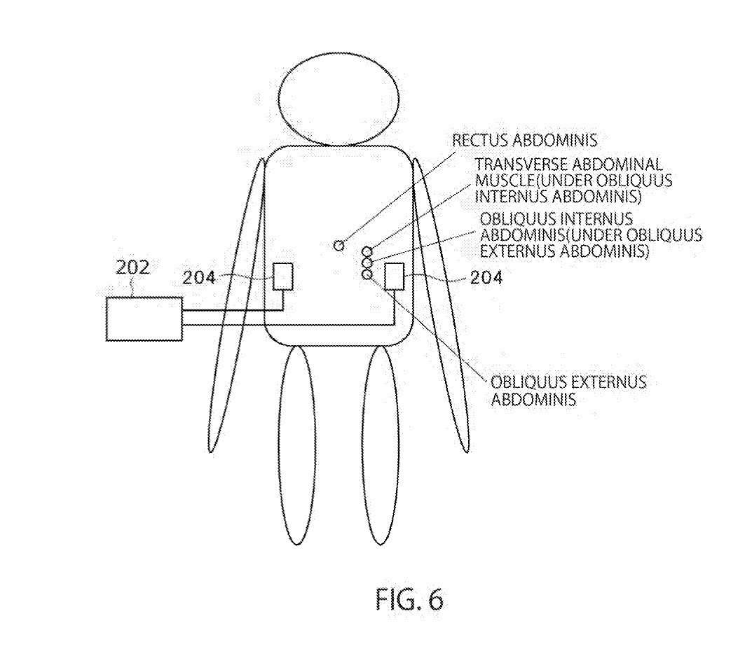

Next, a relation between the contraction of the abdominal muscle and the suppression of the motion of the diaphragm will be explained based on FIG. 6, with reference to FIG. 4. FIG. 6 is a schematic diagram showing the position of the abdominal muscle related to the respiration and the disposed position of the electrode units 204. As shown in FIG. 6, the muscle of the abdomen related to the respiratory motion, that is, the abdominal muscle is roughly configured by four muscles: a rectus abdominis, an obliquus externus abdominis, an obliquus internus abdominis and a transverse abdominal muscle. In the case in which the electrode units 204 are disposed at positions allowing the maintenance electric current to be transmitted to the rectus abdominis, the obliquus externus abdominis, the obliquus internus abdominis, the transverse abdominal muscle and the like and the maintenance electric current is given, the contraction of these muscle tissues is maintained and the movement of the diaphragm is suppressed. The suppression of the movement of the diaphragm will be explained below.

These four muscles are muscles that work at the time of a deep expiration. That is, these are not used in the respiration at rest. The rectus abdominis, which is a muscle running longitudinally from the breast bone to the pubic bone, functions in the flexion of the body, and works so as to place internal organs at predetermined positions at the time of the contraction. Further, the obliquus externus abdominis is used when the body is laid crosswise or is bent, and works so as to assist the rectus abdominis. Furthermore, the obliquus internus abdominis is a muscle that runs obliquely from the pelvis to the rib and that is positioned under the obliquus externus abdominis. The above-described obliquus externus abdominis and obliquus internus abdominis form a cross-coupled shape, and works so as to assist the work of the rectus abdominis. The transverse abdominal muscle is a muscle that runs on the outer side of the abdominal wall and that is positioned at a deep part of the obliquus internus abdominis. At the time of the contraction, the transverse abdominal muscle increases the abdominal pressure, pushes up the diaphragm, and works so as to expire a breath. As understood from this, at the time of the contraction, the four muscles act so as to push up the diaphragm and place the internal organs at the predetermined positions.

Meanwhile, the respiration at rest is performed by the contraction and relaxation of two muscles: an external intercostal muscle and the diaphragm. The external intercostal muscle contracts at the time of the inspiration of a breath, to expand the rib cage to the outside, increase the negative pressure of the thoracic cavity and inflate the lung. Further, the diaphragm is a muscle related to the respiration motion, is a special muscle for the breath inspiration, and contracts at the time of the Inspiration, together with the external intercostal muscle. Further, the diaphragm is a domed muscle membrane in which the head side is the peak, and the periphery of the diaphragm is fixed to the abdominal wall. Therefore, when the diaphragm contracts, the peak of the domed diaphragm moves in a direction allowing the departure from the head side, so that the whole is flattened.

At the time of expiring a breath, that is, at the time of the expiration, the external intercostal muscle and the diaphragm are relaxed. The lung has a characteristic of contracting by itself. Therefore, when these muscles are relaxed, the lung contracts by the contracting force and expires a breath. When the diaphragm is relaxed, the peak of the domed diaphragm rises to the head side again, and the air quantity in the lung decreases.

As understood from this, when the abdominal muscle is contracted by transmitting the maintenance electric current in a state in which a breath is expired at rest, a force that is not applied in the normal respiration at rest is artificially applied to the diaphragm and the internal organs. Therefore, for example, when the maintenance electric current is applied to the abdominal muscle at a timing in the range in which the value of the respiratory waveform shown in FIG. 4 is equal to or less than the threshold value, the diaphragm is pushed to the head side by the push-up force, because the diaphragm is relaxed. It is thought that the pushed diaphragm stops at a position where the push-up force balances with the natural contraction force of the lung itself. In this case, while the contraction of the abdominal muscle is maintained, the abdominal muscle forces act so as to continue to push up the diaphragm, and therefore, the movement of the diaphragm is suppressed. Here, the increase in the time of the stop of the diaphragm allows the increase in the time of the irradiation with the therapeutic beam, and further enhances the therapy efficiency.

Meanwhile, the subject 10 in which the drift phenomenon of the respiration occurs is sometimes irradiated with the therapeutic beam. The position of the stop of the diaphragm at the time of the maximal expiration of such subject 10 tends to shift to a position departing from the head with the lapse of the radiography time. Therefore, it is thought that, also in such subject 10, it is possible to stop the diaphragm such that the position of the diaphragm at the time of the maximal expiration is a more repeatable position by transmitting the maintenance electric current to the abdominal muscle at an appropriate timing. That is, it is thought that the drift phenomenon and the like can be suppressed more because the diaphragm is moved to a further pushed position relative to the position of the diaphragm at the time of the maximal expiration at rest, when the maintenance electric current is applied to the abdominal muscle.

Further, the diaphragm is at the border between the thoracic cavity and the abdominal cavity. The thoracic cavity contains the lung and the heart, and the abdominal cavity contains organs such as the stomach, the pancreas, the gallbladder, the spleen, the liver and the kidney. These organs make the respiratory movement in conjunction with the movement of the diaphragm. Meanwhile, when the maintenance electric current is transmitted to the abdominal muscle as described above, it is possible to stop the diaphragm such that the position of the diaphragm is a more repeatable position. Therefore, it is possible to stop the movements of the organs making the respiratory movement in conjunction with the diaphragm, at more repeatable positions. Thereby, it is possible to set the gate for the therapeutic beam to the affected part target in the organs, to a repeatable position. Therefore, it is possible to further enhance the therapy efficiency. Moreover, since the movement of the diaphragm can be suppressed by transmitting the maintenance electric current to the abdominal muscle, it is possible to further increase the time of the irradiation with the therapeutic beam, and to further enhance the therapy efficiency.

Furthermore, as described above, the diaphragm is a special muscle for the breath inspiration, and contracts at the time of the inspiration, together with the external intercostal muscle. Therefore, for the maintenance in the inspiration state, the maintenance electric current is applied to muscles including the external intercostal muscle and the diaphragm. In this case, since the external intercostal muscle and the diaphragm further contract, the peak of the domed diaphragm moves in a direction allowing the departure from the head side, and the maintenance is performed in a state in which the whole is flattened. Therefore, in the case of the radiography in the inspiration state, it is possible to maintain the inspiration state, by applying the maintenance electric current to the external Intercostal muscle and the diaphragm. In this case, the electrode units 204 are disposed and fixed at skin surface positions allowing the stimulus of muscles including the external intercostal muscle and the diaphragm.

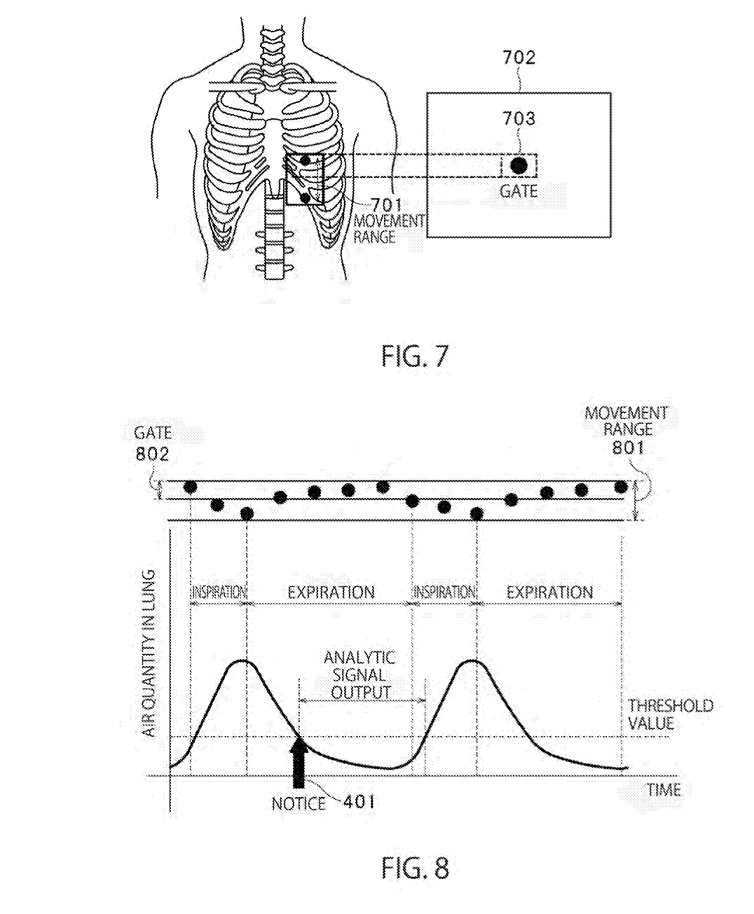

Next, the gate for the therapeutic beam to a tumor that is the affected part target will be explained based on FIG. 7. Here, an example in which the X-ray fluoroscopic radiography is performed while the subject 10 is fixed on the therapy table 108 in a posture when the 4D CT is radiographed in the case in which the subject 10 is fixed on a bed or the like in the radiography of the 4D CT and the tracked object is tracked by the real-time tracking and irradiating system 1 according to the embodiment will be explained. In this case, the positional relation between the position of the affected part target acquired from the 4D CT data and the tumor as the affected part target is repeated as nearly the same relation, when the affected part target is tracked by the real-time tracking and irradiating system 1 according to the embodiment. Further, the explanation will be made using an example in which a tumor at a lower part of the thorax that is the affected part target performs the respiratory movement in accordance with a respiratory cycle.

FIG. 7 is a schematic diagram showing a movement range of a tumor and the position of a gate in a 4D CT. The left diagram of FIG. 7 is a schematic diagram showing a range in which the tumor moves in the 4D CT. As shown in the left diagram of FIG. 7, the tumor developed in the lung field moves in a movement range 701 shown by the arrow in the rectangular box, in accordance with the respiratory cycle. That is, the tumor moves to the vicinity of an uppermost part on the head side, at the time of the maximal expiration, and moves to the vicinity of a lowermost part on the foot side, at the time of the maximal Inspiration. It is said that the respiratory cycle is a frequency of approximately 12 to 20 times/minute in an adult at rest. That is, one respiratory cycle is about 3 to 5 seconds.

Next, the gate for the therapeutic beam will be explained based on the right diagram of FIG. 7. The right diagram of FIG. 7 is a schematic diagram showing a two-dimensional Image 702 obtained based on an electric signal that is obtained in the first X-ray radiographing unit 110A, and a gate position 703 in the two-dimensional image 702. As shown in the right diagram of FIG. 7, the gate position 703 for the therapeutic beam is set based on the position where the tumor moves to the vicinity of the uppermost part. When the tumor is at this position, the position corresponds to the position where the diaphragm at rest is relaxed most. That is, the position where the tumor moves to the vicinity of the uppermost part has a high repeatability, and the tumor is at the position for a longer time.

Further, the real-time tracking and irradiating system 1 is set such that the position where the tumor moves to the vicinity of the uppermost part is radiographed at a nearly central part of each radiographing surface of the first X-ray radiographing unit 110A and the second X-ray radiographing unit 110B. In this way, the settings are performed based on the position of the tumor part obtained in the 4D CT.

Next, a relation between the respiratory waveform and the movement of the tumor that is the affected part target will be explained based on FIG. 8, with reference to FIG. 7. Here, the explanation will be made using the example of the 4D CT and tumor explained in FIG. 7. FIG. 8 is a schematic diagram showing the relation between the respiratory waveform and the movement of the tumor that is the affected part target. The abscissa Indicates time, the ordinate in the upper diagram indicates a range 801 corresponding to the movement range of the tumor shown in FIG. 7, and the ordinate in the lower diagram indicates the air quantity in the lung. Further, a gate 802 corresponds to the ordinate of the gate 703 in FIG. 7. As shown in FIG. 8, the tumor making the respiratory movement moves in conjunction with the respiratory waveform. That is, the tumor moves to the foot side with the progression of the inspiration, and the tumor moves to the head side with the progression of the expiration. Particularly, in a period from a middle time of the expiration to the start of the inspiration, the movement quantity of the tumor is smaller than in the other period. That is, the gate 802 for the therapeutic beam is set in a range in which the movement quantity per time of the tumor that is the affected part target is smaller than that in the other period. As described above, the range in which the analyzing unit 234 in the embodiment outputs the analytic signal is set roughly to the range of the position of the affected part target in the gate 802. As described above, this period includes the period in which the diaphragm at rest is relaxed most, leading to the increase in a repeatability in which the affected part target exists in the gate even when the respiratory cycle is repeated.

As understood from these, based on the respiratory waveform, the threshold value is set in an analytic function, in consideration of the repeatability of the position of the affected part target. Further, based on the threshold value, the maintenance electric current is transmitted to the abdominal muscle, and the movement of the diaphragm is suppressed, leading to the increase in the time during which the affected part target is in the gate 802.

Further, based on the output of the analytic signal from the analyzing unit 234, the respiratory waveform displaying unit 210 displays the respiratory waveform and the mark 401. Thereby, the subject 10 can monitor the respiratory state while performing the free respiration, and can perform the electric stimulus of the abdominal muscle tissue for a predetermined time, for his or her own convenience, at the timing when the affected part target is in the gate 802. Further, it is possible to temporarily suppress the movement of the diaphragm, which is the main factor of the respiratory movement, by utilizing a physiological response by which the muscle tissue cannot be relaxed by his or her intention during the electric stimulus. Thereby, with a good repeatability, it is possible to temporarily stop the affected part target in the gate 802 for the therapeutic beam, and to perform a respiratory synchronization allowing a more efficient irradiation with the therapeutic beam.

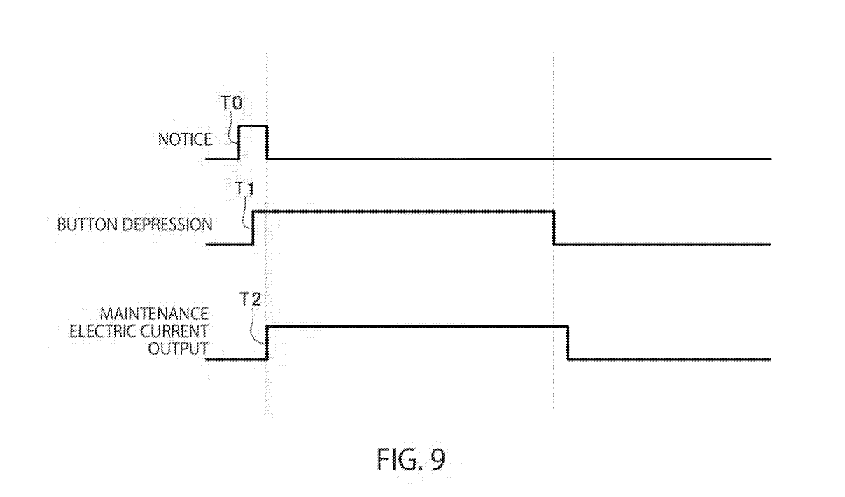

Next, a behavior of the electric current generating apparatus 200 according to the embodiment will be explained based on FIG. 9. Here, the explanation will be made using an example in which the first mode is selected through the inputting unit 218 and the maintenance electric current is output while the push button unit 206 is depressed.

FIG. 9 is a diagram showing a control timing of the electric current generating apparatus 200. The abscissa indicates time, and the ordinate indicates an ON state and an OFF state. As shown in FIG. 9, at time “T0”, the notifying unit 236 notifies the subject 10 that the subject 10 is in the previously set respiratory state, based on the analytic signal from the analyzing unit 234.

Next, at time “T1”, the push button unit 206 is depressed in accordance with the operation by the subject 10. Based on the depression, under the control by the controlling unit 220, the electric current generating unit 222, at time “T2”, starts the generation of the maintenance electric current for maintaining the contraction of the abdominal muscle related to the motion of the diaphragm, so that the maintenance electric current is output from the electrode units 204 that transmit the maintenance electric current to the abdominal muscle. Thereby, the movement of the diaphragm is stopped by the operation of the push button unit 206 by the subject 10. That is, in this case, the movement of the diaphragm is temporarily suppressed in the previously set respiratory state.

Next, the depression of the push button unit 206 is canceled in accordance with the operation by the subject, and based on the cancel, the electric current generating unit 222 stops the generation of the maintenance electric current in accordance with the control by the controlling unit 220. Thus, the abdominal muscle is relaxed to return to the normal respiratory state at rest, in accordance with the operation of the push button unit 206 by the subject 10.

As described above, in the electric current generating apparatus 200 according to the embodiment, the maintenance electric current generated by the electric current generating unit 160 is transmitted from the electrode units 204 to the abdominal muscle related to the motion of the diaphragm, in accordance with the depression operation of the push button unit 206 by the subject 10. Therefore, it is possible to maintain the contraction of the abdominal muscle and suppress the motion of the diaphragm in accordance with the operation by the subject 10. Furthermore, since the notifying unit 236 gives a notice when the subject 10 is in the previously set respiratory state, it is possible to suppress the motion of the diaphragm in the previously set respiratory state, in accordance with the operation by the subject 10.

### Second Embodiment

A second embodiment is different from the first embodiment in that a radiographing system according to the second embodiment further includes a CT (Computed Tomography) system 1000 using a CT 500 in addition to the real-time tracking and irradiating system 1 according to the first embodiment. The difference from the first embodiment will be explained below.

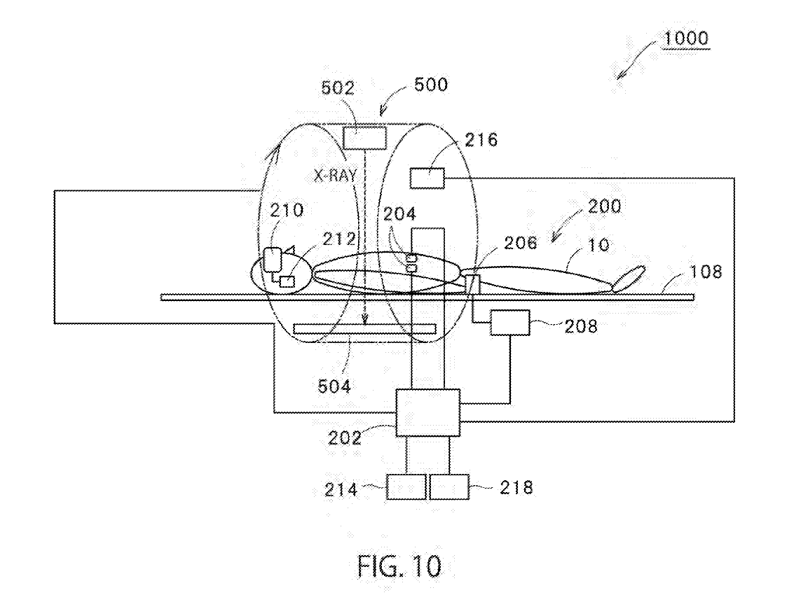

A whole configuration of the CT system 1000 according to the second embodiment will be explained based on FIG. 10, with reference to FIG. 2. FIG. 10 is a block diagram for explaining the whole configuration of the CT system 1000 according to the embodiment. Identical numerals are assigned to the same constituents as those in the first embodiment, and the explanation is omitted.

As shown in FIG. 10, the CT system 1000 according to the embodiment is a system that performs the CT scan radiography of the subject by CT radiography and that suppresses the respiratory action of the subject 10 by electric stimulus, and is configured to include the electric current generating apparatus 200 and the CT 500. That is, there is a difference in that the medical imager according to the real-time tracking and irradiating system 1 is the FPD (Flat Panel Detector) with an indirect conversion scheme but the medical imager according to the CT system 1000 is the CT 500. Here, the real-time tracking and irradiating system 1 is disposed in a different examination room from the CT system 1000.

In the radiography with use of the CT 500, generally, the radiography is performed in the inspiration state. That is, the radiography is performed in a state in which a deep respiration is performed and the respiration is stopped. Therefore, the electrode units 204 are disposed and fixed at skin surface positions allowing the stimulus of muscles including the external intercostal muscle and the diaphragm. That is, the radiography is performed in a state in which a deep respiration is performed and the external Intercostal muscle and the diaphragm are contracted by the maintenance electric current output by the electrode units 204.

The electric current output controlling unit 226 performs a control to output the maintenance electric current to the electrode units 204, when the subject 10 is in a previously set respiratory state. More specifically, the electric current output controlling unit 226 controls the pulse generating unit 224 such that the maintenance electric current is generated, when the value indicating the air quantity in the lung of the subject is equal to or more than a second threshold value.

Further, the electric current output controlling unit 226 alters the previously set respiratory state, depending on the type of the medical imager that is used for the radiography of the subject. More specifically, depending on the type of the medical imager that is used for the radiography of the subject 10, the electric current output controlling unit 226 may adopt a previously set first respiratory state when the value indicating the air quantity in the lung of the subject is equal to or less than a first threshold value, or may adopt a previously set second respiratory state when the value indicating the air quantity in the lung of the subject 10 is equal to or more than the second threshold value.

For example, in the case in which the medical imager is an FPD (Flat Panel Detector), the electric current output controlling unit 226 adopts the previously set first respiratory state when the value indicating the air quantity in the lung of the subject is equal to or less than the first threshold value. In this case, the electrode units 204 are disposed and fixed at positions allowing the maintenance electric current to be transmitted to the rectus abdominis, the obliquus externus abdominis, the obliquus internus abdominis, the transverse abdominal muscle and the like.

Further, for example, in the case in which the medical imager is the CT 500, the previously set second respiratory state is adopted when the value indicating the air quantity in the lung of the subject 10 is equal to or more than the second threshold value. In this case, the electrode units 204 are disposed and fixed at skin surface positions allowing the stimulus of muscles including the external Intercostal muscle and the diaphragm. The first threshold value and the second threshold value herein are values that are experimentally set.

The CT 500 images the affected part target in the subject 10 using X-rays, and obtains a three-dimensional Image of the affected part target. That is, the CT 500 is configured to include an X-ray generating unit 502 and a sensor 504.

The X-ray generating unit 502 generates an X-ray pulse. The sensor 504 converts the X-ray having penetrated the subject 10, into an image signal. The X-ray generating unit 502 and the sensor 504 rotate in the orientation of the arrow about an unillustrated rotating shaft, and acquire the image signal of the subject from the directions of 360 degrees.

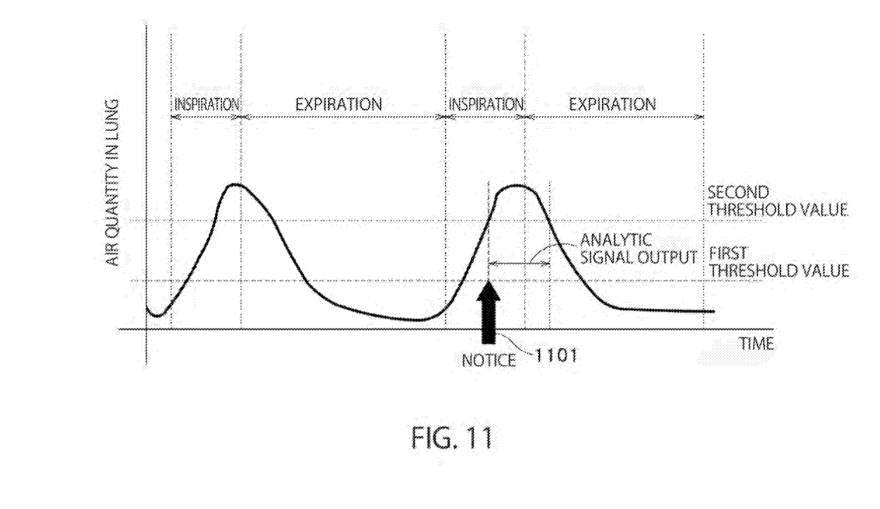

Next, a process in the analyzing unit 234 according to the embodiment will be explained based on FIG. 11, with reference to FIG. 2. FIG. 11 is a schematic diagram showing a time series change in the air quantity in the lung and an output range of the analytic signal according to the second embodiment. The abscissa in FIG. 11 indicates time, and the ordinate in the figure indicates the air quantity in the lung.

As shown in FIG. 11, the analyzing unit 234 outputs the analytic signal, when the value of the respiratory waveform generated by the respiratory waveform generating unit 232 is equal to or more than the second threshold value, that is, when the value indicating the air quantity in the lung is equal to or more than the second threshold value. The second threshold value is set, for example, based on the value indicating the air quantity in the lung at the time of the maximal inspiration. For example, the second threshold value is set to a value of 80% of the value of the respiratory waveform at the time of the maximal inspiration.

Further, a marker 1101 is displayed on the respiratory waveform displaying unit 210, based on the timing when the analyzing unit 234 starts the output of the analytic signal.

Furthermore, when the above-described second mode or third mode is selected, the electric current generating unit 202 starts the generation of the maintenance electric current, by the control from the electric current output controlling unit 226, based on the timing when the analyzing unit 234 starts the output of the analytic signal. In this case, the timing when the CT 500 starts the radiography is also based on the timing when the analyzing unit 234 starts the output of the analytic signal. Moreover, the time when the electric current generating unit 202 ends the generation of the maintenance electric current is based on the timing when the CT 500 ends the radiography.

As described above, in the electric current generating apparatus 200 according to the embodiment, the electric current output controlling unit 226 performs the control to output the maintenance electric current to the electrode units 204, when the subject 10 is in the previously set respiratory state. Thereby, it is possible to perform the CT radiography while maintaining the previously set respiratory state. In this case, since the body movement due to the respiration is suppressed, it is possible to obtain a CT image in which motion artifact is decreased.

### Third Embodiment

A third embodiment is different from the second embodiment in that a radiographing system according to the third embodiment further includes a plain radiographing system 1200 in addition to the real-time tracking and irradiating system 1 and the CT system 1000 according to the second embodiment. The difference from the second embodiment will be explained below.

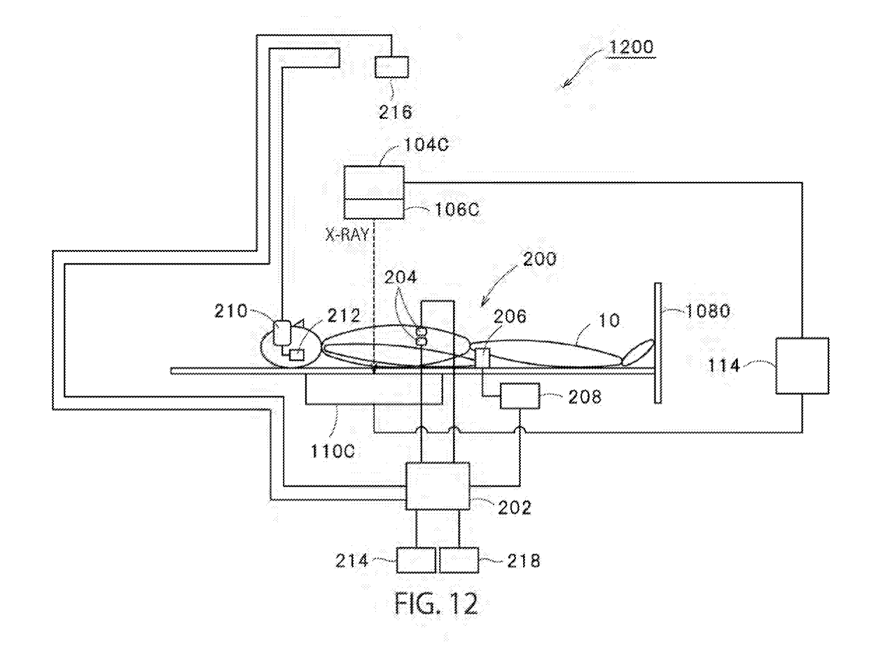

A whole configuration of the plain radiographing system 1200 according to the embodiment will be explained based on FIG. 12, with reference to FIG. 2. FIG. 12 is a block diagram for explaining the whole configuration of the plain radiographing system 1200 according to the third embodiment. Identical numerals are assigned to the same constituents as those in the first embodiment. As shown in FIG. 12, the plain radiographing system 1200 according to the embodiment is a system that performs the plain radiography of the subject and that suppresses the respiratory action of the subject 10 by electric stimulus, and is configured to include a third X-ray tube holding unit 104C, a third collimator unit 106C, a third X-ray radiographing unit 110C, the synchronization controlling unit 114, the electric current generating apparatus 200 and a supporting unit 1080.

The third X-ray tube holding unit 104C, which has the same configuration as that of the first X-ray tube holding unit 104A, irradiates the subject 10 with X-rays. Furthermore, the third collimator unit 106C, which has the same configuration as that of the first collimator unit 106A, restricts the irradiation range of the X-ray that is generated by the third X-ray tube holding unit 104C. The third X-ray radiographing unit 110C, which has the same configuration as that of the first X-ray radiographing unit 110A, converts the X-ray quantity of the X-ray having penetrated the subject 10, into an electric signal, and outputs it. Here, the medical imager is the third X-ray radiographing unit 110C. As described above, the third X-ray radiographing unit 110C, for example, is any one of an FPD and Color I.I.

The supporting unit 1080 supports the third X-ray radiographing unit 110C. Here, an example of the radiography (AP radiography) from the thoracic side to the dorsal is shown. The orientation in the radiography is not limited to this, and a PA radiography may be adopted.

In the plain radiography, generally, the radiography is performed in the inspiration state. Therefore, the electrode units 204 are disposed and fixed at skin surface positions allowing the stimulus of muscles including the external intercostal muscle and the diaphragm.

The electric current output controlling unit 226 performs a control to output the maintenance electric current to the electrode units 204, when the subject 10 is in a previously set respiratory state. More specifically, the electric current output controlling unit 226 makes the electric current generating unit 202 output the maintenance electric current, when the value indicating the air quantity in the lung of the subject is equal to or more than a second threshold value.

Further, the electric current output controlling unit 226 alters the previously set respiratory state, depending on the radiography purpose of the medical imager that is used for the radiography of the subject. More specifically, depending on the radiography purpose of the medical imager that is used for the radiography of the subject 10, the electric current output controlling unit 226 may adopt a previously set first respiratory state when the value indicating the air quantity in the lung of the subject is equal to or less than a first threshold value, or may adopt a previously set second respiratory state when the value indicating the air quantity in the lung of the subject 10 is equal to or more than the second threshold value.

For example, in the case in which the medical imager tracks the position of the affected part target making the respiratory movement, the electric current output controlling unit 226 adopts the previously set first respiratory state when the value indicating the air quantity in the lung of the subject is equal to or less than the first threshold value. Further, for example, in the case in which the medical imager performs the plain radiography, the previously set second respiratory state is adopted when the value indicating the air quantity in the lung of the subject 10 is equal to or more than the second threshold value. The first threshold value and the second threshold value herein are values that are experimentally set.

Next, an exemplary process in the analyzing unit 234 according to the embodiment will be explained based on FIG. 11, with reference to FIG. 2. Similarly to the second embodiment, the analyzing unit 234 outputs a second analytic signal, when the value of the respiratory waveform generated by the respiratory waveform generating unit 232 is equal to or more than the second threshold value, that is, when the value indicating the air quantity in the lung is equal to or more than the second threshold value. The second threshold value is set, for example, based on the value indicating the air quantity in the lung at the time of the maximal inspiration. For example, the second threshold value is set to a value of 80% of the value of the respiratory waveform at the time of the maximal inspiration.

Further, the marker 1101 is displayed on the respiratory waveform displaying unit 210, based on the timing when the analyzing unit 234 starts the output of the analytic signal.

Furthermore, when the above-described second mode or third mode is selected, the electric current generating unit 202 starts the generation of the maintenance electric current, by the control from the electric current output controlling unit 226, based on the timing when the analyzing unit 234 starts the output of the analytic signal. In this case, the timing when the third X-ray tube holding unit 104C starts the X-ray irradiation is also based on the timing when the analyzing unit 234 starts the output of the analytic signal.

As described above, in the electric current generating apparatus 200 according to the embodiment, the electric current output controlling unit 226 performs the control to output the maintenance electric current to the electrode units 204, when the subject 10 is in the previously set respiratory state. Thereby, it is possible to perform the plain radiography while maintaining the previously set respiratory state. In this case, since the body movement due to the respiration is suppressed, it is possible to obtain a plain radiographic image in which motion artifact is decreased.

### Fourth Embodiment

An X-ray irradiating apparatus according to a fourth embodiment makes a difference between the X-ray irradiation state when the maintenance electric current is being transmitted to the subject and the X-ray irradiation state when the maintenance electric current is not being transmitted to the subject, and further decreases the integrated dose of the X-ray with which the subject is irradiated. The detail will be explained below.

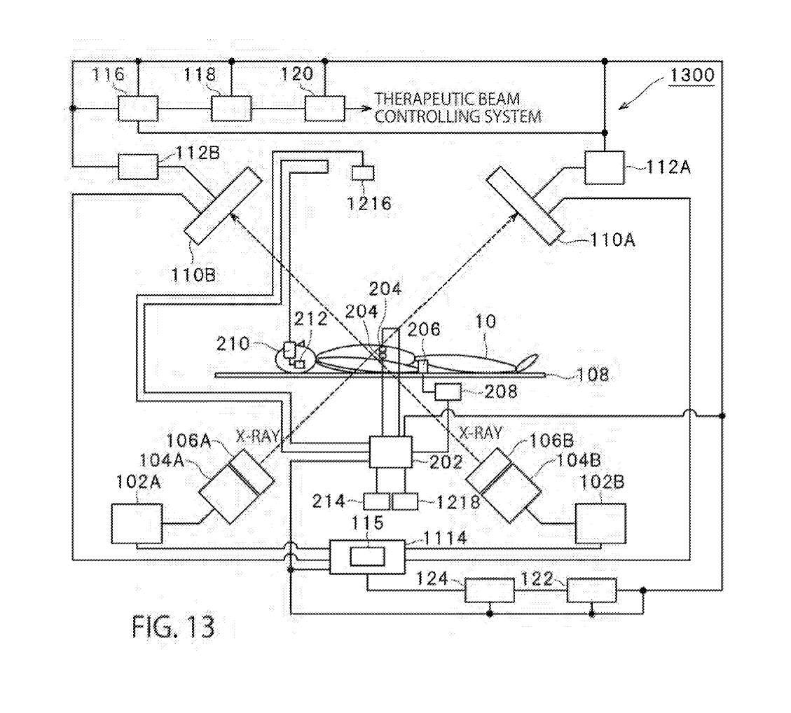

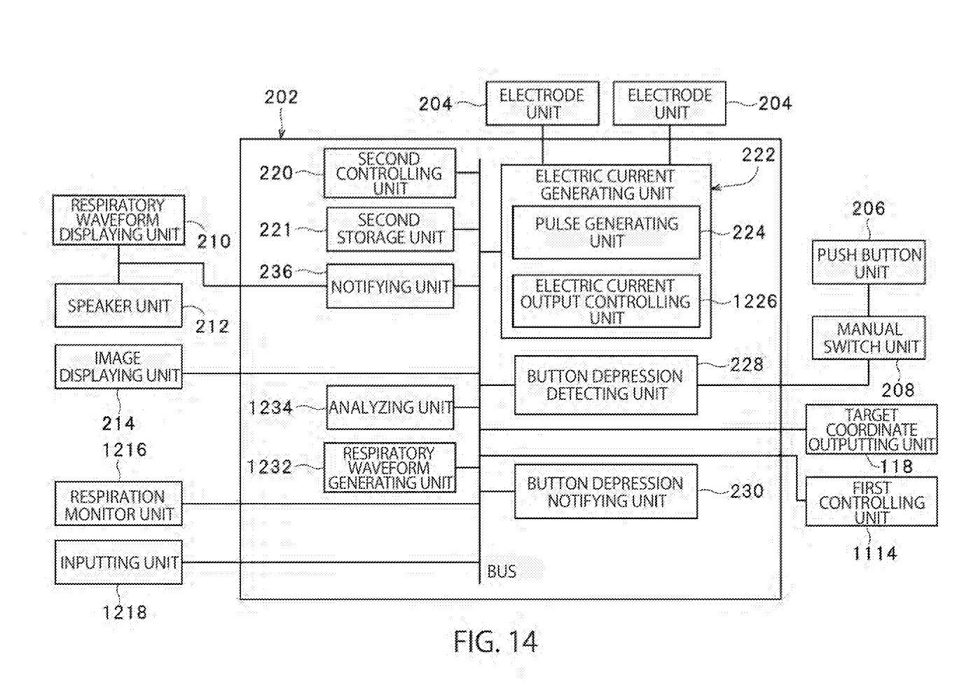

A whole configuration of an X-ray irradiating apparatus 1300 according to the embodiment will be explained based on FIG. 13 and FIG. 14. FIG. 13 is a block diagram for explaining the whole configuration of the X-ray irradiating apparatus 1300 according to the fourth embodiment. As shown in FIG. 13, the X-ray irradiating apparatus 1300 according to the embodiment is an apparatus that Irradiates the affected part target with X-rays and that suppresses the movement of the affected part target by electric stimulus. There is a difference from the first embodiment in that a first controlling unit 1114 performs different controls between the X-ray irradiation state when the maintenance electric current is being transmitted to the subject 10 and the X-ray irradiation state when the maintenance electric current is not being transmitted to the subject 10. Identical numerals are assigned to the same constituents as those in the first embodiment, and the explanation is omitted.

That Is, the X-ray irradiating apparatus 1300 is configured to include the first high-voltage pulse generating unit 102A, the second high-voltage pulse generating unit 102B, the first X-ray tube holding unit 104A, the second X-ray tube holding unit 104B, the first collimator unit 106A, the second collimator unit 106B, the therapy table 108, the first X-ray radiographing unit 110A, the second X-ray radiographing unit 110B, the first 2D image outputting unit 112A, the second 2D image outputting unit 112B, the first controlling unit 1114, a first storage unit 115, the 3D image outputting unit 116, the target coordinate outputting unit 118, the irradiation permission judging unit 120, a position acquiring unit 122, a setting unit 124, an electric current generating body unit 202, the electrode units 204, the push button unit 206, the manual switch unit 208, the respiratory waveform displaying unit 210, the speaker unit 212, the image displaying unit 214, a respiration monitor unit 1216 and an Inputting unit 1218.

The first controlling unit 1114 controls each constituent unit of the X-ray irradiating apparatus 1300. That is, the first controlling unit 1114 is configured by a CPU, for example, and can control each constituent unit by the execution of a program. The first storage unit 115 stores a control program that is executed by the first controlling unit 1114, and provides a work area in the program execution.

The first controlling unit 1114 performs a control to synchronize the generation timings of the high-voltage pulses in the first high-voltage pulse generating unit 102A and the second high-voltage pulse generating unit 102B. Moreover, the first controlling unit 1114 performs a control to synchronize the imaging timings of the first X-ray radiographing unit 110A and the second X-ray radiographing unit 110B with the generation timings of the high-voltage pulses.

Further, the first controlling unit 1114 controls the intensity of the high-voltage pulses generated by the first high-voltage pulse generating unit 102A and the second high-voltage pulse generating unit 102B, the generation frequency and the pulse width of the high-voltage pulses, in accordance with a signal that is input from the exterior. That is, the first controlling unit 1114 controls the intensity, generation frequency and irradiation time of the X-ray that is emitted by the first X-ray tube holding unit 104A and the second X-ray tube holding unit 104B.

The position acquiring unit 122 acquires the position of the affected part target corresponding to the respiratory state of the subject 10, based on multiple X-ray images resulting from imaging the affected part target of the subject 10 in a time series manner. That is, the position acquiring unit 122 acquires the position of the affected part target, based on first image data and second image data obtained in a time series manner from the first 2D image outputting unit 112A and the second 2D image outputting unit 112B respectively.

The setting unit 124 sets the gate that is the irradiation range for the therapeutic beam, to the position of the affected part target in a previously set respiratory state of the subject 10. That is, based on the position of the affected part target acquired by the position acquiring unit 122, the setting unit 124 sets the gate, on the basis of the position of the affected part target in a state in which the diaphragm has been relaxed.

Here, in the embodiment, the first high-voltage pulse generating unit 102A and the first X-ray tube holding unit 104A configure a first X-ray irradiating unit, the second high-voltage pulse generating unit 102B and the second X-ray tube holding unit 104B configure a second X-ray irradiating unit, the first high-voltage pulse generating unit 102A, the second high-voltage pulse generating unit 102B, the first X-ray tube holding unit 104A and the second X-ray tube holding unit 104B configure an X-ray irradiating unit, the first X-ray radiographing unit 110A and the first 2D image outputting unit 112A configure a first X-ray imaging unit, the second X-ray radiographing unit 110B and the second 2D image outputting unit 112B configure a second X-ray imaging unit, the first X-ray radiographing unit 110A, the second X-ray radiographing unit 110B, the first 2D image outputting unit 112A and the second 2D image outputting unit 112B configure an X-ray imaging unit, and the target coordinate outputting unit 118 configures a position detecting unit.

The respiration monitor unit 1216 acquires a measurement signal related to the respiratory waveform, from the subject 10, and outputs it to the electric current generating body unit 202. For example, the measurement signal indicates the height of the abdomen. In the respiration monitor unit 1216, a non-contact sensor and a contact sensor can be used. For example, as the non-contact sensor, an infrared sensor, an ultrasonic sensor, an electric wave sensor, a laser sensor and the like can be used in the respiration monitor unit 1216. That is, the non-contact sensor measures the respiratory state of the subject 10, using one of a light wave, a sound wave and an electric wave.

On the other hand, as the contact sensor, a piezoelectric sensor, a strain gauge sensor, a servo sensor and the like can be used in the respiration monitor unit 1216. Generally, depending on the therapeutic area, the contact sensor sometimes interferes with the irradiation or the fluoroscopy, and is easily influenced by radioactive rays. Therefore, in the example of the embodiment, the non-contact sensor is used.

Further, as the measurement signal, the respiration monitor unit 1216 may acquire the ventilation flow quantity in the lung field of the subject 10. The respiratory waveform herein means the time series change in the air quantity in the lung field. That is, the respiratory waveform is the time series change in the integrated value of the ventilation flow quantity. In the embodiment, the respiration monitor unit 1216 configures a measuring unit.

Furthermore, the respiration monitor unit 1216 may acquire measurement signals related to the respiratory waveform, from multiple sites of the subject 10, and may output them to the electric current generating body unit 202. That is, the respiration monitor unit 1216 acquires multiple measurement signals related to the respiratory waveform, from some of the thorax, the abdomen, the dorsal, the nostril and the mouth.

The inputting unit 1218 inputs an intensity signal indicating the intensity of the maintenance electric current, in accordance with the operation by the operator.

The intensity of the maintenance electric current that is output by the electric current generating body unit 202 is controlled in accordance with the intensity signal.

Further, the inputting unit 1218 inputs a selection signal for selecting an operation mode of transmitting the maintenance electric current to the subject 10, in accordance with the operation by the operator. The selection signal is output to the electric current generating body unit 202, so that the generation of the maintenance electric current is controlled. That is, the inputting unit 1218 is used for selecting one of a zero mode of outputting the maintenance electric current from the electrode units 204 when the subject 10 is in a previously set respiratory state regardless of whether the push button unit 206 is depressed, a first mode of outputting the maintenance electric current from the electrode units 204 when the subject 10 is depressing the push button unit 206, and a second mode of restricting or stopping the output of the maintenance electric current from the electrode units 204 when the subject 10 is depressing the push button unit 206 but the subject 10 is not in the previously set respiratory state.

Furthermore, the inputting unit 1218 inputs a selection signal for selecting the mode of the analyzing process of obtaining an analytic signal that is used for outputting for the maintenance electric current, in accordance with the operation by the operator. That is, the selection signal is used for selecting an A mode or a B mode.

Next, a configuration of the electric current generating body unit 202 will be explained based on FIG. 14, with reference to FIG. 13. FIG. 14 is a block diagram for explaining the configuration of the electric current generating body unit 202 according to the fourth embodiment. As shown in FIG. 14, the electric current generating body unit 202 is configured to include a second controlling unit 220, a second storage unit 221, the electric current generating unit 222, the button depression detecting unit 228, the button depression notifying unit 230, a respiratory waveform generating unit 1232, an analyzing unit 1234, and the notifying unit 236.

In the embodiment, the first controlling unit 1114 and the second controlling unit 220 configure a controlling unit, and the first controlling unit 1114 and the second controlling unit 220 may be integrally configured.

The electric current generating unit 222 generates the maintenance electric current for maintaining the contraction of the muscle by electric stimulus.

That is, the electric current generating unit 222 is configured to include the pulse generating unit 224 and an electric current output controlling unit 1226.

The electric current output controlling unit 1226 controls the pulse generating unit 224. That is, the electric current output controlling unit 1226 controls the generation of the pulsed electric current and the intensity of the pulsed electric current in the pulse generating unit 224, in accordance with the action mode selected by the selection signal from the inputting unit 1218. Further, the electric current output controlling unit 1226 outputs an alteration signal for altering the irradiation frequency of the X-ray, to the first controlling unit 1114, based on the timing of the generation of the pulsed electric current.

When the depression signal is detected, the button depression detecting unit 228 outputs an output signal. That is, the button depression detecting unit 228 continuously outputs the output signal to the electric current output controlling unit 1226, during a period in which the depression signal is being detected. When one of the first mode and the second mode is selected, the electric current output controlling unit 1226 performs such a control that the pulse generating unit 224 generates the maintenance electric current, in response to the input of the output signal.

When one of the first mode and the second mode is selected, the button depression notifying unit 230 generates a picture signal indicating the depression state of the button, in accordance with the input of the output signal of the button depression detecting unit 228. Then, the button depression notifying unit 230 outputs the picture signal to the image displaying unit 214, and thereby, displays a picture indicating the depression state of the button, on the image displaying unit 214.

Here, the relation between the time series change in the height of the abdomen and the respiratory waveform is the same as that in FIG. 3. Therefore, the detail of a generation process in the respiratory waveform generating unit 1232 will be explained with reference to FIG. 3 and FIG. 14. The respiratory waveform generating unit 1232 generates the respiratory waveform, based on the information about the height of the abdomen that is acquired by the respiration monitor unit 1216, and the like.

The respiratory waveform generating unit 1232 generates the respiratory waveform, using a previously obtained relation between the time series change in the height of the abdomen and the time series change in the air quantity in the lung field. The generation of the respiratory waveform is the same as the above-described process in the respiratory waveform generating unit 232. That is, the respiratory waveform generating unit 1232 generates the respiratory waveform, using the previously obtained relation between the time series change in the height of the abdomen and the time series change in the air quantity in the lung field. The detailed process has been described above, and therefore, the explanation is omitted.

Further, the respiratory waveform generating unit 1232 may generate the respiratory waveform, using measurement signals related to respiratory waveforms acquired from multiple sites of the subject 10 in the respiration monitor unit 1216. For example, a new respiratory waveform may be generated by an arithmetic process for obtaining the average value of the value of a respiratory waveform based on the height of the abdomen and the value of a respiratory waveform based on the height of the thorax. Thereby, even when a peculiar change, for example, an abnormal behavior or a noise is made in one respiratory waveform, it is possible to suppress the degree of the peculiar change. Further, the differential value of the respiratory waveform with respect to time change, that is, the change quantity in the value of the respiratory waveform with respect to time may be calculated, and a value of a respiratory waveform for a site with a change quantity exceeding a predetermined value may be excluded in the arithmetic process for obtaining the average value. In this case, it is possible to decrease the influence of the respiratory waveform for the site with the peculiar change.

As described above, the relation between the time series change in the height of the abdomen and the time series change in the air quantity in the lung can be obtained based on the information of the 4D CT image (Four-Dimensional Computed Tomography). The detailed process has been described above, and therefore, the detailed explanation is omitted.

Again, as shown in FIG. 14, the analyzing unit 1234 outputs an analytic signal, when the subject 10 is in a previously set respiratory state, based on one of the respiratory waveform generated by the respiratory waveform generating unit 1232 and the three-dimensional coordinates of the affected part target output by the target coordinate outputting unit 118. When the respiratory waveform has a previously set first phase, the analyzing unit 1234 outputs an X-ray irradiation signal for the instruction of the X-ray irradiation, as the analytic signal, to the first controlling unit 1114. That is, when the air quantity in the lung field is equal to or less than a previously set first threshold value “Th1”, as a first phase, the analyzing unit 1234 starts the output of the X-ray irradiation signal to the first controlling unit 1114.

Further, as the analyzing method for obtaining a maintenance electric current output signal that is the analytic signal for the instruction of the output of the maintenance electric current, the analyzing unit 1234 has two modes, that is, the A mode and the B mode. The A mode, for example, is intended for a subject 10 having a high repeatability of the respiratory waveform. That is, the analyzing unit 1234 outputs the maintenance electric current output signal, based on the respiratory waveform.

The B mode, for example, is intended for a subject 10 having a low repeatability of the respiratory waveform, and the maintenance electric current output signal is output based on the positional information of the affected part target. That is, the analyzing unit 1234 outputs the maintenance electric current output signal to the electric current output controlling unit 1226, at the timing when the affected part target enters the gate.

Here, the low repeatability of the respiratory waveform means that the position of the affected part target varies every respiratory cycle or the respiratory waveform varies every respiratory cycle even through the phase is identical.

Here, the repeatability of the respiratory waveform can be judged, for example, based on the information of a preliminarily radiographed 4D CT.

Next, the analyzing process when the A mode is selected will be explained in detail. When the respiratory waveform has a previously set second phase, the analyzing unit 1234 outputs the maintenance electric current output signal, as the analytic signal, to the electric current output controlling unit 1226. That is, when the air quantity in the lung field is equal to or less than a previously set threshold value “Th2” (Th1≧Th2), as the second phase, the analyzing unit 1234 starts the output of the analytic signal to the electric current output controlling unit 1226. In this case, the analytic signal may be continuously output while the air quantity in the lung field is equal to or less than the previously set threshold value “Th2”. Alternatively, the analytic signal may be continuously output for a previously set time. In the case in which the analytic signal is continuously output for a previously set time, the period in which the maintenance electric current is transmitted to the subject 10 is 0.1 seconds to 3 seconds, for example, and may be set in the therapy plan. Here, the timing when the second phase is generated and the timing when the affected part target enters the gate are previously set so as to correspond to each other.

The threshold value “Th2” is set, for example, based on the information of a preliminarily radiographed 4D CT, and “Th2” is set to a value of 20% of the maximum value of the respiratory waveform, for example. The respiratory state of the subject 10 in the period of the threshold value “Th2” or less is a state in which the diaphragm has been relaxed, and a state in which almost all the air in the lung to be expired at rest has been expired. That is, based on the value of the respiratory waveform at the maximal expiration, the analyzing unit 1234 outputs the maintenance electric current output signal Indicating the previously set respiratory state.

On the other hand, the threshold value “Th1” is set, for example, based on the repeatability of the respiratory waveform of the subject 10. That is, in the case in which the repeatability of the respiratory waveform is high, for example, “Th1” may be the same value as “Th2”. In this case, the X-ray irradiation signal is output at the timing when the maintenance electric current output signal is output. Thereby, at the timing when the affected part target enters the gate that is the Irradiation range for the therapeutic beam, the maintenance electric current is output and the X-ray is emitted. Therefore, when the affected part target does not exist in the gate, the X-ray with which the subject 10 is irradiated is further suppressed, and therefore, it is possible to further decrease the integrated quantity of the X-ray with which the subject 10 is irradiated.

Further, the timing of the first phase may be set so as to be earlier by time “T5” than the timing of the second phase. The time “T5” can be set depending on the repeatability of the respiratory waveform. That is, as the repeatability decreases, the time “T5” is set to a longer time, and therefore, the value of the threshold value “Th1” is set to a higher value. On the other hand, as the repeatability increases, the time “T5” can be set to a shorter time, and therefore, it is possible to further decrease the Integrated quantity of the X-ray with which the subject 10 is irradiated.

Furthermore, when the A mode is selected, the output of the maintenance electric current output signal may be stopped if the affected part target Is not in the gate at the timing of the second phase. That is, the analyzing unit 1234 stops the output of the maintenance electric current output signal, when the three-dimensional coordinates of the affected part target obtained from the target coordinate outputting unit 118 are not in the gate at the timing when the maintenance electric current output signal is output. Thereby, the maintenance electric current is prevented from being output to the subject 10 at a mismatched timing.

Meanwhile, the time “T5” is set to a time range allowing the normal process in the irradiation permission judging unit 120. Therefore, even when the output of the maintenance electric current output signal is stopped, the irradiation with the therapeutic beam can be performed in a state in which the maintenance electric current is not transmitted.

Further, when the A mode is selected, the output time of the X-ray irradiation signal is a period after the timing of the generation of the first phase and before the lapse of the predetermined time “T5” from the end of the output of the maintenance electric current output signal. In the case in which the repeatability of the respiratory waveform is high, it is possible to set “T5” to zero, and it is possible to equalize the output time of the X-ray irradiation signal and the output time of the maintenance electric current output signal. That is, it Is possible to equalize the irradiation time of the X-ray and the output time of the maintenance electric current.

Next, the analyzing process when the B mode is selected will be explained in detail. Because of the correspondence to the case in which the repeatability of the respiratory waveform is low, the time “T5” from the timing of the first phase to the timing of the second phase may be set to a longer time. As described above, the time “T5” can be set to a longer time as the repeatability of the respiratory waveform decreases. Similarly to conventional apparatuses, the time “T5” may be the period of the therapy using the therapeutic beam. That is, in this case, the X-ray is emitted over the entire period of the therapy using the therapeutic beam.

The analyzing unit 1234 outputs the maintenance electric current output signal to the electric current output controlling unit 1226, at the timing when the affected part target enters the gate, based on the three-dimensional coordinates of the affected part target output by the target coordinate outputting unit 118. Thereby, even when the repeatability of the respiratory waveform is low, it is possible to output the maintenance electric current to the subject 10, at the timing when the affected part target enters the gate.

When the B mode is selected, the maintenance electric current output signal may be continuously output while the affected part target is in the gate. Alternatively, the maintenance electric current output signal may be continuously output for a previously set time. In the case in which the analytic signal is continuously output for a previously set time, the period in which the maintenance electric current is transmitted to the subject 10 is 0.1 seconds to 3 seconds, for example, and can be set in the therapy plan. Further, when the B mode is selected, the output time of the X-ray irradiation signal is a period after the timing of the generation of the first phase and before the lapse of predetermined time “T6” from the end of the output of the maintenance electric current output signal. Here, the predetermined time “T6” is a time from the timing of the generation of the first phase to the output of the maintenance electric current output signal. As understood from this, whichever of the A mode and the B mode is selected, it is possible to decrease the integrated dose of the X-ray to the subject 10, depending on the repeatability of the respiratory waveform.

Furthermore, the analyzing unit 1234 generates a notice signal as the analytic signal. That is, a third phase is a timing when the air quantity in the lung field becomes equal to or less than a previously set third threshold value “Th3”, and the analyzing unit 1234 outputs the notice signal at the timing of the third phase. The timing of the third phase is set so as to be earlier by time “T7” than the timing of the second phase. The time “T7” is set to a time that allows the transmission of the maintenance electric current to be previously recognized in consideration of the time for the depression of the push button unit 206.

Again, as shown in FIG. 14, the notifying unit 236 gives a notice that the subject 10 is in the previously set respiratory state. That is, the notifying unit 236 displays the marker and the corresponding respiratory waveform on the respiratory waveform displaying unit 210, based on the analytic signal output by the analyzing unit 1234. That is, the notifying unit 236 displays the respiratory waveform and the like on the respiratory waveform displaying unit 210, in accordance with the notice signal input from the analyzing unit 1234. On the other hand, when the B mode is selected, the notifying unit 236 displays the respiratory waveform and the like on the respiratory waveform displaying unit 210, in accordance with the maintenance electric current output signal input from the analyzing unit 1234.

Further, the speaker unit 212 outputs a sound signal at the timing of the display of the marker. Thereby, the subject 10 is notified that the respiratory state of the subject 10 is the previously set respiratory state. The notifying unit 236 may display the marker during a predetermined period from the timing when the analyzing unit 1234 outputs the analytic signal.

The whole configuration of the X-ray irradiating apparatus 1300 according to the embodiment has been explained above. Next, a control behavior of the electric current output controlling unit 1226 will be explained.

When the zero mode is selected, the electric current output controlling unit 1226 controls the pulse generating unit 224 such that the maintenance electric current is generated, in response to the maintenance electric current output signal from the analyzing unit 1234. That is, the electric current output controlling unit 1226 controls the pulse generating unit 224 such that the maintenance electric current is generated, when the maintenance electric current output signal is being input regardless of whether the push button unit 206 is depressed.

When the first mode is selected, the maintenance electric current can be output from the electrode units 204, by the depression of the push button unit 206 by the subject 10. That is, while the output signal is being input from the button depression detecting unit 228, the electric current output controlling unit 1226 controls the pulse generating unit 224 such that the maintenance electric current is generated. Here, the subject 10 can depress the push button unit 206 at the timing of the previously set respiratory state, in accordance with the notice from the notifying unit 236. As understood from this, the electric current output controlling unit 1226 controls the pulse generating unit 224 in response to the output signal, regardless of whether the maintenance electric current output signal is input.

When the second mode is selected, the control behavior as the first mode is performed, and therewith, in the case in which the subject 10 is not in the previously set respiratory state, the output of the maintenance electric current from the electrode units 204 is restricted or prohibited. That is, the electric current output controlling unit 1226 controls the pulse generating unit 224 such that the maintenance electric current Is generated, in the case in which the output signal is being input and where the maintenance electric current output signal is being input. Thereby, it is possible to avoid the motion of the diaphragm from being suppressed in the case in which the subject 10 is not in the previously set respiratory state.

Further, when the second mode is selected, by the depression of the push button unit 206, the maintenance electric current is automatically output from the electrode units 204 in the case of being in the previously set respiratory state. Therefore, the subject 10 need not match the push timing of the push button unit 206 with the notice from the notifying unit 236.

As understood from this, when the first mode or the second mode is selected, the subject 10 can give priority to his or her physical condition. That is, for example, when the rhythm of the respiration is unstable, the subject 10 need not push the push button unit 206, and can avoid the maintenance electric current from being output into the body. Therefore, the subject 10 can reflect his or her intention or condition in the output of the maintenance electric current from the electrode units 204.

Further, the maintenance electric current may be continuously output from the electrode units 204 while the subject 10 is depressing the push button unit 206. Alternatively, the time during which the maintenance electric current is continuously output from the electrode units 204 may be previously set to a predetermined time. In the case of setting the predetermined time, the electric current output controlling unit 1226 stops the generation of the maintenance electric current after the lapse of the predetermined time, even when the depression of the push button unit 206 is continued. The predetermined time can be set based on the physical condition of the subject 10, the above-described respiratory waveform before the start of the therapy, and the like. Here, it is preferable that the predetermined time be set in the therapy plan, because of the association with the time of the irradiation with the therapeutic beam.

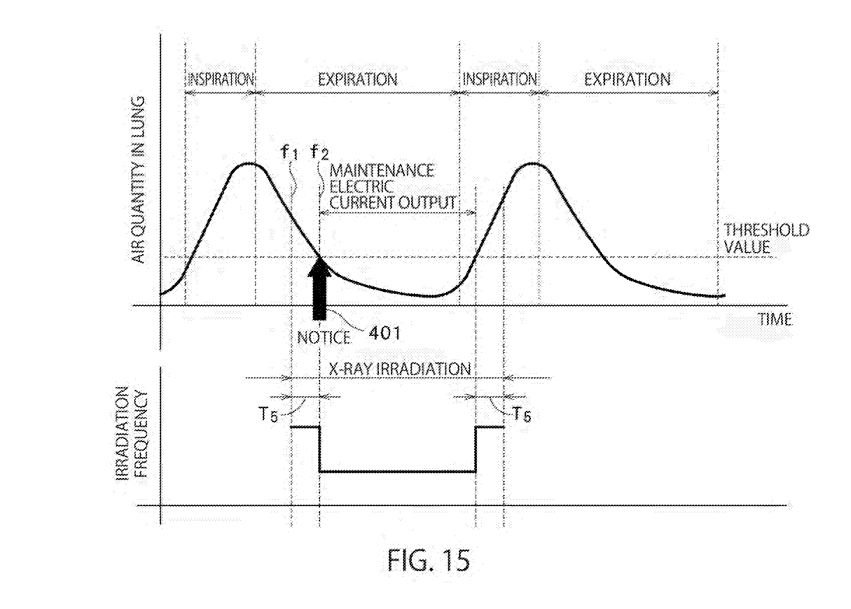

Next, an exemplary control behavior based on the output signal of the analyzing unit 1234 will be explained based on FIG. 15. FIG. 15 is a schematic diagram showing a time series change in the air quantity in the lung and an output timing of the analytic signal according to the fourth embodiment.

The abscissa in FIG. 15 indicates time, and the ordinate in the upper diagram Indicates the air quantity in the lung. Further, the ordinate in the lower diagram indicates the irradiation frequency of the X-ray. Here, a case in which the above-described A mode and zero mode are selected will be explained. Further, the explanation will be made using an example in which a tumor at a lower part of the thorax that is the affected part target performs the respiratory movement in accordance with a respiratory cycle.

As shown in FIG. 15, at a first phase “f1” of the respiratory waveform, an X-ray irradiation signal is input from the analyzing unit 1234 to the first controlling unit 1114. Thereby, the first X-ray tube holding unit 104A and the second X-ray tube holding unit 104B start the X-ray irradiation. In synchronization with the X-ray irradiation, the first X-ray radiographing unit 110A images the first image data, and the second X-ray radiographing unit 110B images the second image data. Subsequently, based on the image data, the target coordinate outputting unit 118 outputs the three-dimensional coordinates of the affected part target to the analyzing unit 1234. Then, at a second phase “f2” of the respiratory waveform, the analyzing unit 1234 outputs the maintenance electric current output signal to the electric current output controlling unit 1226, if the three-dimensional coordinates are in the range of the gate. That is, at the second phase “f2” as the previously set respiratory state of the subject 10, the maintenance electric current output signal is output with the condition that the three-dimensional coordinates are in the range of the gate. Here, since the three-dimensional coordinates are in the range of the gate, the maintenance electric current output signal is continuously output while the air quantity in the lung field is equal to or less than the previously set threshold value.

As understood from this, the first controlling unit 1114 performs a control to start the X-ray irradiation, to the first high-voltage pulse generating unit 102A and the second high-voltage pulse generating unit 102B, based on the information of the respiratory waveform. That is, the first controlling unit 1114 performs a control to start the X-ray irradiation, depending on the respiratory state of the subject. Thereby, when the affected part target is at a position away from the gate, an unnecessary X-ray irradiation is not performed to the subject 10, and therefore, the integrated dose of the X-ray is further decreased.

Further, the analyzing unit 1234 outputs a notice signal to the notifying unit 236. Thereby, the notifying unit 236 displays the marker 401 and the corresponding respiratory waveform on the respiratory waveform displaying unit 210.

Next, the electric current output controlling unit 1226 outputs an alteration signal for decreasing the X-ray irradiation frequency, to the first controlling unit 1114, based on the timing of the generation of the maintenance electric current. The alteration signal is continuously output to the first controlling unit 1114, while the maintenance electric current is being output. Subsequently, the first controlling unit 1114 performs a control to decrease the X-ray irradiation frequency, to the first high-voltage pulse generating unit 102A and the second high-voltage pulse generating unit 102B. That is, the first controlling unit 1114 performs a control to decrease the X-ray irradiation frequency when the maintenance electric current is being transmitted to the subject 10, relative to the X-ray irradiation frequency when the maintenance electric current is not being transmitted to the subject 10. As understood from this, the first controlling unit 1114 performs the control to alter the X-ray irradiation state, to the first high-voltage pulse generating unit 102A and the second high-voltage pulse generating unit 102B, based on the information of the respiratory waveform. That is, the first controlling unit 1114 performs the control to alter the X-ray irradiation state, depending on the respiratory state of the subject.

Furthermore, when the alteration signal is input, the first controlling unit 1114 performs a control to decrease the X-ray intensity and increase the irradiation time, to the first high-voltage pulse generating unit 102A and the second high-voltage pulse generating unit 102B. That is, the first controlling unit 1114 performs a control to decrease the X-ray intensity and increase the irradiation time when the maintenance electric current is being transmitted to the subject 10 relative to the X-ray intensity and irradiation time when the maintenance electric current Is not being transmitted to the subject 10, to the first high-voltage pulse generating unit 102A and the second high-voltage pulse generating unit 102B. For example, the X-ray irradiation is controlled such that the “mas” value of the X-ray with which the subject 10 is irradiated is a constant value. Thereby, it is possible to decrease the irradiation intensity of the X-ray, and therefore, it is possible to decrease the load on the X-ray tube and the like.

Next, the first controlling unit 1114 performs a control to restore the irradiation frequency of the X-ray, to the first high-voltage pulse generating unit 102A and the second high-voltage pulse generating unit 102B, based on the timing when the input of the alteration signal is stopped. That is, the first controlling unit 1114 performs a control to continue the X-ray irradiation, to the first high-voltage pulse generating unit 102A and the second high-voltage pulse generating unit 102B, for the same time as the time “T5” from the timing of the first phase to the timing of the second phase.

As understood from this, the X-ray irradiation is not performed until the first phase in the previously set respiratory state of the subject 10. Therefore, it is possible to suppress an unnecessary X-ray irradiation, and it is possible to decrease the Integrated dose of the X-ray, compared to the conventional case in which the X-ray irradiation is continued over the entire period. Moreover, while the maintenance electric current is being transmitted to the subject 10, the irradiation frequency of the X-ray is further decreased, and therefore, it is possible to further decrease the integrated dose of the X-ray. For example, it is possible to decrease the integrated dose of the X-ray to the subject 10, to one-tenth or less, compared to the conventional case in which the X-ray irradiation is continued over the entire period.

The maintenance electric current generated by the electric current generating unit 222 according to the embodiment is the same as the pulsed electric current with a pulse form explained based on FIG. 5. Further, the reaction of the muscle tissue of the subject 10 to electric stimulus is the same as the above. Therefore, the detailed explanation here is omitted.

The disposed position of the electrode units 204 according to the embodiment is the same as the content explained based on FIG. 6 described above. That is, when the electrode units 204 are disposed at positions allowing the maintenance electric current to be transmitted to the rectus abdominis, the obliquus externus abdominis, the obliquus internus abdominis, the transverse abdominal muscle and the like, the contraction of these muscle tissues is maintained, and the movement of the diaphragm at the time of the expiration is suppressed.

Further, when the electrode units 204 are disposed at skin surface positions allowing the stimulus of muscles including the external intercostal muscle and the diaphragm, the contraction of these muscle tissues is maintained, and the movement of the diaphragm at the time of the inspiration is suppressed. The explanation of the suppression of the movement of the diaphragm is the same as the above, and therefore, the explanation here is omitted.

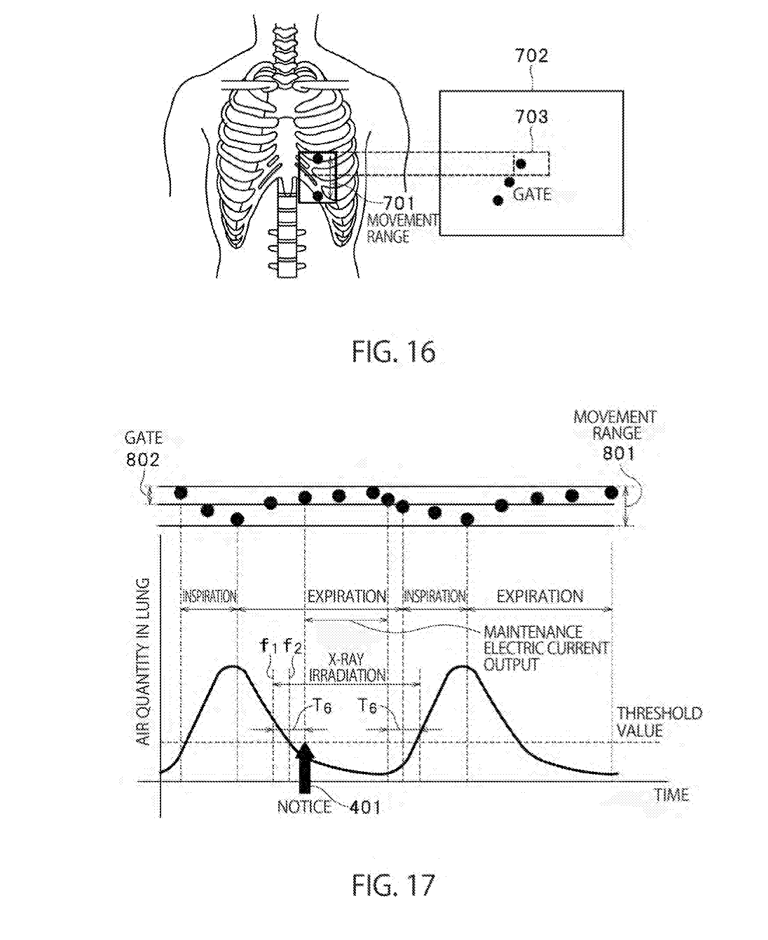

Next, the gate for the therapeutic beam to a tumor that Is the affected part target will be explained based on FIG. 16. FIG. 16 is a schematic diagram showing a movement range of the tumor and the position of a gate in a 4D CT according to the fourth embodiment. Here, an example in which the subject 10 is fixed on the bed 108 and is preliminarily radiographed and the X-ray fluoroscopic radiography is performed while the subject 10 is fixed on the therapy table 108 in the posture at the time of the preliminary radiography when the tracked object is tracked by the X-ray irradiating apparatus 1300 according to the embodiment will be explained. In this case, the positional relation between the position of the preliminarily radiographed affected part target and the tumor as the affected part target is repeated as nearly the same relation, when the affected part target is tracked by the X-ray irradiating apparatus 1300 according to the embodiment. Further, the explanation will be made using an example in which a tumor at a lower part of the thorax that is the affected part target performs the respiratory movement in accordance with a respiratory cycle.

The left diagram of FIG. 16 is a schematic diagram showing a range in which the tumor that is the affected part target moves in the 4D CT. As shown in the left diagram of FIG. 16, the tumor developed in the lung field moves in the movement range 701 shown by the arrow in the rectangular box, in accordance with the respiratory cycle. That is, the tumor moves to the vicinity of an uppermost part on the head side, at the time of the maximal expiration, and moves to the vicinity of a lowermost part on the foot side, at the time of the maximal inspiration. It is said that the respiratory cycle is a frequency of approximately 12 to 20 times/minute in an adult at rest. That is, one respiratory cycle is about 3 to 5 seconds.

Next, the gate for the therapeutic beam will be explained based on the right diagram of FIG. 16. The right diagram of FIG. 16 is a schematic diagram showing the two-dimensional image 702 obtained based on an electric signal that is obtained in the first X-ray radiographing unit 110A, and a tumor position acquired by the position acquiring unit 122. As shown in the right diagram of FIG. 16, the position acquiring unit 122 acquires the two-dimensional position of the tumor from each of multiple two-dimensional images 702 acquired in a time series manner.

The setting unit 124 sets the gate 703 for the therapeutic beam, based on the position where the tumor moves to the vicinity of the uppermost part. The case in which the tumor is positioned in the gate 703 corresponds to a state in which the diaphragm at rest is relaxed most. That is, the gate 703 is set so as to correspond to the position of the tumor in the state in which the diaphragm of the subject 10 is relaxed. Therefore, the repeatability in which the tumor enters the gate 703 in the respiratory cycle is high, and the time during which the tumor is positioned is longer.

Similarly, the gate position is set based on the multiple two-dimensional images acquired by the second X-ray radiographing unit 110B in a time series manner. Further, the setting unit 124 sets a three-dimensional gate region corresponding to the set two-dimensional gate regions, in the irradiation permission judging unit 120.

Furthermore, the X-ray irradiating apparatus 1300 is set such that the position where the tumor moves to the vicinity of the uppermost part is radiographed at a nearly central part of each radiographing surface of the first X-ray radiographing unit 110A and the second X-ray radiographing unit 110B. In this way, the settings are performed based on the position of the tumor part that is the affected part target.

Here, the gate only needs to be set at a position allowing a high repeatability and an increase in the time during which the tumor as the affected part target is positioned. Therefore, the gate may be set so as to correspond to the position of the tumor in the state of the maximal expiration when the diaphragm moves most to the foot side. In this case, the setting of the first phase, second phase and third phase in the analyzing unit 1234 can be performed on the basis of the state of the maximal expiration.

Next, an exemplary control behavior based on the output signal from the analyzing unit 1234 will be explained based on FIG. 17, with reference to FIG. 16. FIG. 17 is a schematic diagram showing a relation between the respiratory waveform and the movement of the tumor that is the affected part target according to the fourth embodiment. The abscissa indicates time, the ordinate in the upper diagram indicates the range 801 corresponding to the movement range of the tumor shown in FIG. 16, and the ordinate in the lower diagram indicates the air quantity in the lung. Further, the gate 802 corresponds to the ordinate of the gate 703 in FIG. 16. Here, the explanation will be made using the example of the tumor explained in FIG. 16. Further, a case in which the above-described B mode and the second mode are selected will be explained.

As shown in FIG. 17, at the first phase “f1” of the respiratory waveform, the X-ray irradiation signal is input to the first controlling unit 1114. Thereby, the first X-ray tube holding unit 104A and the second X-ray tube holding unit 104B start the X-ray irradiation. In synchronization with the X-ray irradiation, the first X-ray radiographing unit 110A images the first image data, and the second X-ray radiographing unit 110B images the second image data. Subsequently, based on the image data, the target coordinate outputting unit 118 outputs the three-dimensional coordinates of the affected part target, to the analyzing unit 1234. Then, the analyzing unit 1234 outputs the maintenance electric current output signal to the electric current output controlling unit 1226, when the three-dimensional coordinates are in the range of the gate 802 and the depression signal indicating the depression of the push button unit 206 is input. That is, the analyzing unit 1234 outputs the maintenance electric current output signal to the electric current output controlling unit 1226, at the timing when the affected part target enters the gate 802, based on the three-dimensional coordinates of the affected part target output by the target coordinate outputting unit 118. Thereby, the pulse generating unit 224 outputs the pulsed electric current as the maintenance electric current, to the electrode units 204, in accordance with the control by the electric current output controlling unit 1226. In this way, when the three-dimensional coordinates are in the range of the gate 802 as the previously set respiratory state of the subject 10, the maintenance electric current output signal is output with the condition that the depression signal is input.

Next, the electric current output controlling unit 1226 outputs the alteration signal for decreasing the irradiation frequency of the X-ray, to the first controlling unit 1114, based on the timing of the generation of the maintenance electric current. The alteration signal is continuously output to the first controlling unit 1114 while the maintenance electric current is being output. Subsequently, the first controlling unit 1114 performs the control to decrease the irradiation frequency of the X-ray, to the first X-ray tube holding unit 104A and the second X-ray tube holding unit 104B. That is, the first controlling unit 1114 decreases the X-ray irradiation frequency when the maintenance electric current is being transmitted to the subject 10, relative to the X-ray irradiation frequency when the maintenance electric current is not being transmitted to the subject 10. In this case, the first controlling unit 1114 performs the control to decrease the intensity of the X-ray and increase the irradiation time relative to the X-ray when the maintenance electric current is not being transmitted to the subject 10.

Further, the analyzing unit 1234 outputs the maintenance electric current output signal to the notifying unit 236. Thereby, the notifying unit 236 displays the marker 401 and the corresponding respiratory waveform on the respiratory waveform displaying unit 210. Since the B mode is selected, the notifying unit 236 performs the displaying process, using the maintenance electric current output signal.

Next, the analyzing unit 1234 stops the output of the maintenance electric current output signal, at the timing when the affected part target moves out of the gate 802, based on the three-dimensional coordinates of the affected part target output by the target coordinate outputting unit 118. Subsequently, the first controlling unit 1114 performs the control to restore the irradiation frequency of the X-ray, to the first high-voltage pulse generating unit 102A and the second high-voltage pulse generating unit 102B, based on the timing when the input of the alteration signal is stopped. That is, the first controlling unit 1114 performs the control to continue the X-ray irradiation, to the first high-voltage pulse generating unit 102A and the second high-voltage pulse generating unit 102B, for the same time as the time “T6” from the timing of the first phase to the timing of the output of the maintenance electric current output signal.

As understood from this, even when the repeatability of the respiratory waveform is low, it is possible to output the maintenance electric current in the case in which the affected part target is in the range of the gate 802. Furthermore, the X-ray irradiation is not performed until the first phase in the previously set respiratory state of the subject 10. Therefore, it is possible to suppress an unnecessary X-ray irradiation, and it is possible to decrease the integrated dose of the X-ray to the subject 10, compared to the conventional case in which the X-ray irradiation is continued over the entire period. Moreover, while the maintenance electric current is being transmitted to the subject 10, the irradiation frequency of the X-ray is further decreased, and therefore, it is possible to further decrease the integrated dose of the X-ray.

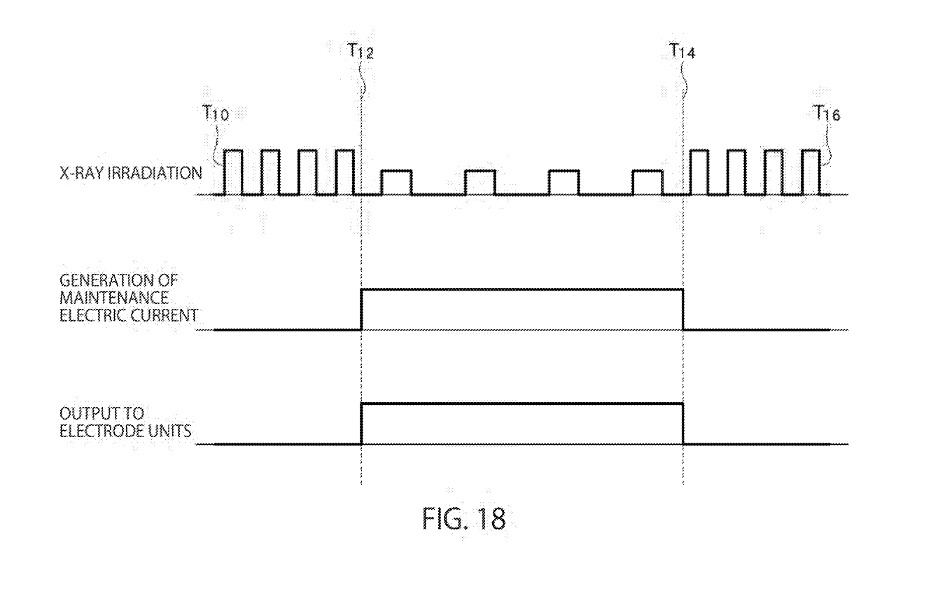

Next, a behavior of the X-ray irradiating apparatus 1300 according to the embodiment will be explained based on FIG. 18. Here, a case in which the A mode and the second mode are selected will be explained.

FIG. 18 is a diagram showing a control timing of the X-ray irradiating apparatus 1300. The abscissa indicates time, and the ordinate indicates an ON state and an OFF state. As shown in FIG. 18, the analyzing unit 1234 outputs the X-ray irradiation signal to the first controlling unit 1114, at time “T10”, which is the timing of the first phase of the respiratory waveform. Thereby, the first X-ray tube holding unit 104A and the second X-ray tube holding unit 104B start the X-ray irradiation.

Next, when the depression signal indicating the depression of the push button unit 206 is input, the analyzing unit 1234 outputs the maintenance electric current output signal to the electric current output controlling unit 1226, at time “T12”, which is the timing of the second phase of the respiratory waveform. Thereby, the electric current generating unit 222 starts the output of the pulsed electric current as the maintenance electric current. At the same time, the maintenance electric current is output to the electrode units 204. At time “T12”, the electric current output controlling unit 1226 outputs the alteration signal to the first controlling unit 1114, based on the generation of the maintenance electric current in the electric current generating unit 222. Further, at the time “T12”, the first controlling unit 1114, to which the alteration signal is input, performs the control to alter the frequency, intensity and Irradiation time of the X-ray, to the first high-voltage pulse generating unit 102A and the second high-voltage pulse generating unit 102B.

Next, at time “T14”, the first controlling unit 1114, for which the input of the alteration signal is stopped, performs the control to restore the X-ray irradiation state to the same state 10 as the state from the time “T10” to the time “T12”, to the first high-voltage pulse generating unit 102A and the second high-voltage pulse generating unit 102B. Then, the first controlling unit 1114 performs the control to perform the X-ray irradiation for the same time as the time from the time “T10” to the time “T12” and stop the X-ray irradiation at time “T16”.

As described above, in the X-ray irradiating apparatus 1300 according to the embodiment, the first controlling unit 1114 varies the X-ray irradiation state when the X-ray tube holding units 104A, 104B irradiate the subject, between when the maintenance electric current is being transmitted to the subject 10 and when the maintenance electric current is not being transmitted.

Therefore, it is possible to further decrease the integrated dose of the X-ray with which the subject 10 is irradiated. Furthermore, the first controlling unit 1114 performs the control to start the irradiation of the subject 10 with the X-ray, based on the first phase “f1” of the respiratory waveform, which is the previously set respiratory state of the subject 10. Therefore, it is possible to suppress an unnecessary X-ray irradiation, and it is possible to further decrease the Integrated dose of the X-ray with which the subject 10 is irradiated.

At least a part of the electric current generating apparatus, the real-time tracking and Irradiating system, the CT system, the plain radiographing system and the X-ray irradiating apparatus explained in the above-described embodiments may be configured by hardware, or may be configured by software. In the case of being configured by software, a program for actualizing at least a part of the functions of the electric current generating apparatus, the real-time tracking and irradiating system, the CT system, the plain radiographing system and the X-ray irradiating apparatus may be stored in a recording medium such as a flexible disk and a CD-ROM, and may be read by a computer, to be executed. The recording medium is not limited to a removable disk such as a magnetic disk and an optical disc, and may be a fixed-type recording medium such as a hard disk device and a memory.

Further, the program for actualizing at least a part of the functions of the electric current generating apparatus, the real-time tracking and irradiating system, the CT system, the plain radiographing system and the X-ray irradiating apparatus may be distributed through a communication line (including a wireless communication) such as the Internet.

Moreover, the program may be distributed in an encrypted, modulated or compressed state, through a wired line or wireless line such as the Internet, or while being stored in the recording medium.

The several embodiments of the present invention are explained above. However, the embodiments are presented as examples and are not intended to limit the scope of the invention. These new embodiments can be implemented in other various forms. Various omissions, substitutions, and changes can be made in a range not departing from the gist of the invention. These embodiments and modifications of the embodiments are included in the scope and the gist of the invention and included in the inventions described in claims and a scope of equivalents of the inventions.

1. An X-ray irradiating apparatus capable of transmitting a maintenance electric current that suppresses motion of a diaphragm in a subject, the X-ray irradiating apparatus comprising:
an electric current outputting unit that outputs the maintenance electric current, the maintenance electric current maintaining contraction of a muscle related to the motion of the diaphragm by electric stimulus;
an electrode unit that is disposed on a skin surface of the subject, the electrode unit transmitting the maintenance electric current to the muscle;
an electric current output controlling unit that controls the electric current outputting unit to switch between a state in which the maintenance electric current is output to the electrode unit and a state in which the maintenance electric current is not output to the electrode unit; and
an operating unit that operates the electric current output controlling unit based on an operation signal in response to an operation.

2. The X-ray irradiating apparatus according to claim 1, wherein the electric current output controlling unit controls the electric current outputting unit to output the maintenance electric current to the electrode unit, when the subject is in a previously set respiratory state.

3. The X-ray irradiating apparatus according to claim 2, wherein the previously set respiratory state is altered depending on a type of a medical imager that is used for radiography of the subject.

4. The X-ray irradiating apparatus according to claim 2, wherein the previously set respiratory state is a state that is based on comparison between a value indicating an air quantity in a lung of the subject and a predetermined threshold value.

5. The X-ray irradiating apparatus according to claim 2, wherein depending on type of a medical imager that is used for radiography of the subject, events are changed between a case in which when a value indicating an air quantity in a lung of the subject is equal to or less than a first threshold value, the previously set respiratory state is entered and a case in which when the value indicating the air quantity in the lung of the subject is equal to or more than a second threshold value, the previously set respiratory state is entered.

6. The X-ray irradiating apparatus according to claim 2, further comprising an analyzing unit that outputs an analytic signal based on a respiratory waveform of the subject, the analytic signal indicating that the subject is in the previously set respiratory state,
wherein the electric current output controlling unit makes the electric current outputting unit output the maintenance electric current, based on the analytic signal.

7. The X-ray irradiating apparatus according to claim 2, wherein the previously set respiratory state corresponds to a position where a position of an affected part target enters a gate for a therapeutic beam.

8. The X-ray irradiating apparatus according to claim 1, further comprising a notifying unit that gives a notice that the subject is in a previously set respiratory state.

9. The X-ray irradiating apparatus according to claim 1, wherein the electric current output controlling unit performs the control in accordance with a signal of the operating unit that switches between the state in which the maintenance electric current is output to the electrode unit and the state in which the maintenance electric current is not output to the electrode unit.

10. The X-ray irradiating apparatus according to claim 9, wherein the respiratory waveform generating unit generates a respiratory waveform, using a relation between a time series change in a height of an abdomen and a time series change in an air quantity in a lung field, the relation being previously obtained based on information obtained from a 4D CT image.

11. The X-ray irradiating apparatus according to claim 1, further comprising one of a displaying unit and a sound generating unit,
wherein the notifying unit makes the displaying unit display a marker together with a respiratory waveform of the subject, in the case of comprising the displaying unit, and
the notifying unit makes the sound generating unit generate a sound that is audible for the subject, in the case of comprising the sound generating unit.

12. The X-ray irradiating apparatus according to claim 6, wherein the analyzing unit analyzes a time during which the subject is in the previously set respiratory state, based on a respiratory waveform in the state in which the maintenance electric current is not output from the electrode unit, and the electric current output controlling unit makes the electric current outputting unit output the maintenance electric current during a period that is a predetermined time longer than the time.

13. The X-ray irradiating apparatus according to claim 1,
wherein the electrode unit
is disposed and fixed at a skin surface position allowing stimulus of muscles including a rectus abdominis, an obliquus externus abdominis, an obliquus internus abdominis and a transverse abdominal muscle, in a first medical imager, and
is disposed and fixed at a skin surface position allowing stimulus of muscles including an external intercostal muscle and the diaphragm, in a second medical imager that is different in type from the first medical imager, and
the maintenance electric current is a pulsed electric current, and a generation interval of the pulsed electric current is an interval at which contraction of the muscles is maintained.

14. A real-time tracking and irradiating system comprising a real-time tracking and irradiating apparatus including:
the X-ray irradiating apparatus according to claim 1;
a first X-ray irradiating unit that irradiates the subject with a first X-ray;
a first X-ray imaging unit that images a first X-ray image based on the first X-ray having penetrated the subject;
a second X-ray irradiating unit that irradiates the subject with a second X-ray;
a second X-ray imaging unit that images a second X-ray image based on the second X-ray having penetrated the subject; and
a position detecting unit that detects a position of a tracked object in the subject based on the first X-ray image and the second X-ray image.

15. A control method for an X-ray irradiating apparatus capable of transmitting a maintenance electric current to a subject, the maintenance electric current maintaining contraction of a muscle by electric stimulus, the control method for the X-ray irradiating apparatus comprising:
outputting the maintenance electric current with an electric current outputting unit, the maintenance electric current maintaining contraction of an abdominal muscle related to motion of a diaphragm;
outputting the maintenance electric current to an electrode unit that transmits the maintenance electric current to the abdominal muscle; and
switching between a state in which the maintenance electric current is output to the electrode unit and a state in which the maintenance electric current is not output to the electrode unit, in accordance with an operation of an operating unit by the subject.

16. An X-ray irradiating apparatus capable of transmitting a maintenance electric current to a subject, the maintenance electric current maintaining contraction of a muscle by electric stimulus, the X-ray irradiating apparatus comprising:
an X-ray irradiating unit that irradiates the subject with an X-ray; and
a controlling unit that controls the X-ray irradiating unit such that there is a difference between an Irradiation state of the X-ray when the maintenance electric current is being transmitted to the subject and an Irradiation state of the X-ray when the maintenance electric current is not being transmitted to the subject.

17. The X-ray irradiating apparatus according to claim 16, wherein the controlling unit decreases an irradiation frequency of the X-ray when the maintenance electric current is being transmitted to the subject, relative to an irradiation frequency of the X-ray when the maintenance electric current is not being transmitted to the subject.

18. The X-ray irradiating apparatus according to claim 16, wherein the controlling unit decreases an intensity of the X-ray and increases an irradiation time when the maintenance electric current is being transmitted to the subject, relative to an intensity and irradiation time of the X-ray when the maintenance electric current is not transmitted to the subject.

19. The X-ray irradiating apparatus according to claim 16, wherein the controlling unit causes the irradiation with the X-ray when the maintenance electric current is being transmitted to the subject, and does not cause the irradiation with the X-ray when the maintenance electric current is not being transmitted to the subject.

20. The X-ray irradiating apparatus according to claim 16, further comprising:
a position acquiring unit that obtains a position of an affected part target corresponding to a respiratory state of the subject, based on multiple X-ray images resulting from imaging the affected part target of the subject in a time series manner; and
a setting unit that sets a gate for a therapeutic beam, to a position of the affected part target corresponding to a previously set respiratory state, based on the position of the affected part target corresponding to the respiratory state of the subject.
